(19) United States
(12) Patent Application Publication
(10) Pub. No.: US 2019/0070244 A1
(43) Pub. Date: Mar. 7, 2019

(54) BOYSENBERRY COMPOSITIONS AND METHODS OF PREPARATION AND USE THEREOF

(71) Applicant: The New Zealand Institute for Plant and Food Research Limited, Palmerston North (NZ)

(72) Inventors: Odette M. Shaw, Palmerston North (NZ); Roger D. Hurst, Palmerston North (NZ)

(21) Appl. No.: 16/023,327

(22) Filed: Jun. 29, 2018

Related U.S. Application Data
(60) Provisional application No. 62/527,166, filed on Jun. 30, 2017.

(51) Int. Cl. A61K 36/73; A61P 11/06; A61P 11/08; A61P 9/02; A61K 47/10; A61K 45/06

(52) U.S. Cl. CPC A61K 36/73; A61P 11/06; A61K 45/06; A61P 9/02; A61K 47/10; A61P 11/08

(57) ABSTRACT

The present disclosure encompasses compositions prepared from Boysenberry. Also encompassed are methods of preparing these compositions and methods of using these compositions, in particular, for treating or preventing disorders of the respiratory system, including amongst others: inflammation, asthma, chronic obstructive pulmonary disease, reactive airway disease, airway fibrosis, and airway remodeling.

### FIELD OF THE INVENTION

The present disclosure relates to compositions prepared from Boysenberry. The present invention relates also to methods of preparing such compositions, and methods of using such compositions, including methods of treating or preventing disorders of the respiratory tract, such as inflammatory conditions of the respiratory tract, including asthma, chronic obstructive pulmonary disease, reactive airway disease, airway fibrosis, and airway remodeling and the physiological conditions that lead to these conditions.

### BACKGROUND OF THE INVENTION

Airway remodeling is understood as a progressive and irreversible decline in airway function due to chronic inflammatory processes that result in structural changes in the airway walls (67). Remodeling of the airways may involve all layers of the airway walls and can occur anywhere along the respiratory tract, from the large to the small airways. Remodeling leads to key changes in epithelial tissue (68). Damaged epithelial cells release profibrotic cytokines, including EGF and TGF-β, which leads to fibroblast proliferation, myofibroblast activation, and ultimately to the formation of subepithelial fibrosis (69). Airway smooth muscle hypertrophy and hyperplasia lead to an increase in airway wall thickness. In turn, this leads to accelerated lung function decline and irreversible or only partially reversible airflow obstruction.

It is estimated that 150 million people are affected by asthma worldwide, with a 5-15% prevalence in children (61). The prevalence of COPD is estimated to be between 15-20%, and it is estimated to cause 2.75 million deaths per annum (86). In the case of chronic asthma there is evidence of cumulative tissue remodeling, fibrosis, and consequent loss of lung function (45, 59). Fibrosis and remodeling are also associated with COPD. Remodeling manifests as a progressive increase in symptoms such as dyspnea and a corresponding decrease in bronchodilator responsiveness (67). Current asthma treatments are designed to manage inflammation and mitigate the symptoms and severity of asthma attacks (30, 43). COPD treatments are also designed to control inflammation and improve airflow. However, no asthma or COPD medications are known to prevent airway remodeling (70-74), and there are no current treatments available to prevent aberrant remodeling.

Asthma pathogenesis and lung tissue remodeling have been linked to an increase in profibrotic, arginase-positive, alternatively activated macrophages (AAMs) in the lung (27, 29, 34). However, temporal depletion of macrophage populations in a model of bleomycin-induced pulmonary fibrosis illustrates that lung macrophages may also develop fibrolytic functions that contribute toward the resolution of fibrosis (14).

Mediators of tissue remodeling, such as the matrix metalloproteinases (MMPs), play an important role in regulating fibrosis (5, 7, 8, 10, 38). Of these, MMP-9 is widely reported to increase in conditions of lung inflammation and fibrosis and is associated with improved symptoms in asthma sufferers (25, 32, 33). MMP-9, in concert with other MMPs, exerts fibrolytic activity that leads to the breakdown of denatured collagens that could moderate inappropriate lung remodeling (5, 60). As such, MMP-9 may represent a possible therapeutic target to limit lung damage in chronic asthma as well as other pulmonary diseases.

Large epidemiological studies have found that increased fruit and vegetable consumption correlates with reduced asthma symptoms (39, 46, 47). These population studies have identified foods high in polyphenols such as apples, pears (13, 51, 62), carrots, tomatoes (46-48), and citrus (11) as having inverse correlations with frequency and severity of reported asthma symptoms, in particular wheeze and cough symptoms (11, 13, 46, 47). However, the effect of fruits high in polyphenols on lung fibrosis and tissue remodeling is unknown. To date, no generally successful methods for preventing airway remodeling have been established.

Given the occurrence of respiratory disorders in the population, including asthma, COPD, reactive airway disease, airway fibrosis, and airway remodeling, there is a need for new compositions, particularly compositions derived from natural sources, for restoring and maintaining respiratory health.

### SUMMARY OF THE INVENTION

In one aspect, the invention encompasses a method of treating or preventing inflammation in the respiratory tract, comprising: administering to a subject a composition comprising a Boysenberry concentrate, thereby treating or preventing the inflammation in the respiratory tract in the subject.

Also encompassed is a composition, for example, a nutraceutical composition, comprising a Boysenberry concentrate for treating or preventing inflammation in the respiratory tract in a subject.

In one other aspect, the invention encompasses a method of treating or preventing asthma, comprising: administering to a subject a composition comprising a Boysenberry concentrate, thereby treating or preventing the asthma in the subject.

Also encompassed is a composition, for example, a nutraceutical composition, comprising a Boysenberry concentrate for treating or preventing asthma in a subject.

In yet one other aspect, the invention encompasses a method of treating or preventing chronic obstructive pulmonary disease, comprising: administering to a subject a composition comprising a Boysenberry concentrate, thereby treating or preventing the chronic obstructive pulmonary disease in the subject.

Also encompassed is a composition, for example, a nutraceutical composition, comprising a Boysenberry concentrate for treating or preventing chronic obstructive pulmonary disease in a subject.

In still one other aspect, the invention encompasses a method of treating or preventing aberrant collagen deposition or fibrosis in the respiratory tract, comprising: administering to a subject a composition comprising a Boysenberry concentrate, thereby treating or preventing the aberrant collagen deposition or fibrosis in the respiratory tract of the subject.

Also encompassed is a composition, for example, a nutraceutical composition, comprising a Boysenberry concentrate for treating or preventing aberrant collagen deposition or fibrosis in a subject.

In even one other aspect, the invention encompasses a method of treating or preventing airway remodeling, comprising: administering to a subject a composition comprising a Boysenberry concentrate, thereby treating or preventing the airway remodeling in the subject.

Also encompassed is a composition, for example, a nutraceutical composition, comprising a Boysenberry concentrate for treating or preventing airway remodeling in a subject.

In various aspects:

The composition comprises Boysenberry juice concentrate or Boysenberry powder.

The composition comprises a dosage unit comprising about 5 to about 500 mg total anthocyanins.

The composition is formulated for enteral administration.

The composition is formulated for oral administration.

The composition is formulated as a syrup or as drops.

The composition is formulated as a gel or jelly.

The composition is formulated as a tablet or capsule.

The composition is formulated for administration at a dosage of about 0.1 mg/kg to about 10 mg/kg total anthocyanins/subject's body weight.

The composition is formulated for administration at a dosage of about 10 mg to about 1000 mg total anthocyanins per day.

Alternatively, the dosage is about 10 mg to about 200 mg total anthocyanins per day, or about 50 mg total anthocyanins per day.

The composition comprises added polyphenols.

The composition is formulated for co-administration with a further respiratory aid.

The composition is formulated for co-administration with one or more treatments for a chronic respiratory disorder.

The inflammation is associated with a chronic respiratory disorder.

The inflammation is associated with one or more of: asthma, chronic obstructive pulmonary disease, reactive airway disease, airway fibrosis, and airway remodeling.

The asthma is atopic or non-atopic.

The asthma is associated with airway fibrosis or airway remodeling.

The chronic obstructive pulmonary disease is associated with smoking or pollution.

The chronic obstructive pulmonary disease is associated with airway fibrosis or airway remodeling.

The aberrant collagen deposition or the fibrosis is associated with a chronic respiratory disorder.

The aberrant collagen deposition or the fibrosis is associated with asthma or chronic obstructive pulmonary disease.

The airway remodeling is associated with a chronic respiratory disorder.

The airway remodeling is associated with one or more of: asthma and chronic obstructive pulmonary disease.

In still one further aspect, the invention comprises the use of a composition comprising a Boysenberry concentrate for preparing a nutraceutical composition for:

(i) treating or preventing inflammation in a respiratory tract in a subject;

(ii) treating or preventing asthma in a subject;

(iii) treating or preventing chronic obstructive pulmonary disease in a subject;

(iv) treating or preventing reactive airway disease in a subject;

(v) treating or preventing aberrant collagen deposition in a subject;

(vi) treating or preventing fibrosis in a respiratory tract in a subject;

(vii) treating or preventing airway remodeling in a subject.

In various aspects, the therapeutic use employs the compositions, dosages, and formulations, and relates to the various conditions, as noted above.

The foregoing brief summary broadly describes the features and technical advantages of certain embodiments of the present invention. Further technical advantages will be described in the detailed description of the invention and examples that follows.

Novel features that are believed to be characteristic of the invention will be better understood from the detailed description of the invention when considered in connection with any accompanying figures and examples. However, the figures and examples provided herein are intended to help illustrate the invention or assist with developing an understanding of the invention, and are not intended to limit the invention's scope.

### BRIEF DESCRIPTION OF THE DRAWINGS

FIG. 1A. Therapeutic oral Boysenberry treatment reduces OVA-induced chronic lung inflammation: 6-wk-old male C57B1/6 mice (n=10 per group) were primed ip with OVA/alum (day 0) then challenged i.n. with OVA every 7 days for 10 wk. From weeks 6 to 10 Boysenberry juice was administered orally (gavage) 1 h prior to, and 2 days after, each i.n. OVA challenge. FIG. 1B: representative H&E staining of lung tissue from naive, 10-wk OVA challenge only (OVA), 10-wk OVA challenge with therapeutic Boysenberry (OVA BoysB) treatment, and Boysenberry alone (BoysB)-treated mice. Arrows and * indicate immune cell infiltrate. Magnification X4 (top) and X10 (bottom). FIG. 1C: representative AB-PAS staining of lung tissue. Arrows indicate dark purple mucus-positive bronchioles. Magnification X4 (top) and X20 (bottom). FIG. 1D: total cells per ml BALF and flow cytometric quantification of percentage of eosinophils in BALF following final OVA challenge. **P<0.01, ***P<0.001 (n=10 per group) one-way ANOVA with Tukey's post hoc test compared with naive and OVA challenge with therapeutic Boysenberry treatment and Boysenberry alone-treated mice.

FIG. 2A. Boysenberry treatment increases arginase expression and macrophage accumulation in lung tissue during OVA-induced chronic lung inflammation: representative H&E staining of lung tissue from 10-wk OVA-challenged mice, with and without Boysenberry treatment. Arrows indicate macrophages. Magnification ×100, scale 200 μm. FIG. 2B: representative Western blot analysis of iNOS (135 kDa) and arginase (37 kDa) expression in lung tissue. Noncontiguous bands from the same Western blot are shown. FIG. 2C and FIG. 2D: quantification of iNOS and arginase Western blot signals normalized to β-actin signal. **P<0.01 (n=10 per group) one-way ANOVA with Tukey's post hoc test.

FIG. 3A. Boysenberry treatment increases the accumulation of arginase+ alternatively activated macrophages. Representative immunofluorescent labelling of lung tissue from 10-wk OVA-challenged mice with and with-out Boysenberry treatment: CD68+CD206+ macrophages identified by *. FIG. 3B: CD206+ arginase+ macrophages identified by *. DAPI nuclear stain (dark blue). Magnification ×40, scale 200 um.

FIG. 4A. Boysenberry treatment decreases collagen deposition and increases MMP-9 protein expression in lung tissue during OVA-induced chronic lung inflammation: representative Masson's trichrome staining. Magnification ×40, scale 200 μm. FIG. 4B: hydroxyproline levels (mg/g lung tissue); ***P<0.001 (n=10) one-way ANOVA with Tukey's post hoc test. FIG. 4C: lung TGFβ concentration as determined by ELISA; *P 0.05 (n 10 per group) one-way ANOVA with Tukey's post hoc test. FIG. 4D: Western blot analysis of MMP-9 (pro 105 kDa; active 92 kDa) and TIMP-1 (29 kDa) expression (noncontiguous bands from the same Western blot are shown) in lung tissue from 10-wk OVA-challenged mice with and without Boysenberry treatment. FIG. 4E: ratio of TIMP-1/MMP-9 protein expression normalized to β-actin loading control; **P<0.01 (n=10) one-way ANOVA with Tukey's post hoc test compared with naive and OVA plus Boysenberry treatment.

FIG. 5A. Boysenberry treatment increases MMP-9 expression by alternatively activated macrophages in lung tissue during OVA-induced chronic lung inflammation: DAB labelling of MMP-9+ macrophages (arrows). FIG. 5B: immunofluorescent labelling of CD206+MMP-9+ macrophages (*). DAPI nuclear stain (dark blue). Magnification X40, scale 200 μm.

FIG. 6A. Depletion of lung macrophages reduced the effect of oral Boysenberry treatment on OVA-induced chronic lung inflammation: 6-wk-old male C57B1/6 mice (n=10 per group) were primed ip with OVA/alum (day 0) then challenged i.n. with OVA every 7 days for 5 wk. From weeks 6 to 7 macrophages were depleted using clodronate liposomes (CloLip) the day before Boysenberry juice was administered orally (gavage). FIG. 6B: flow cytometric quantification of percentage of macrophages in BALF following final clodronate macrophage depletion; *P<0.05 (n=10 per group) one-way ANOVA with Tukey's post hoc test. FIG. 6C: hydroxyproline levels (mg/g lung tissue) in the lung; *P<0.05 (n=10 per group) one-way ANOVA with Tukey's post hoc test.

FIG. 7A. Prophylactic oral Boysenberry treatment reduces OVA-induced chronic lung inflammation and collagen deposition: 6-wk-old male C57B1/6 mice (n=10 per group) were primed ip with OVA/alum then challenged i.n. with OVA every 7 days for 5 wk. Boysenberry juice was administered orally (gavage) 1 h prior and 2 days after each i.n. OVA challenge. FIG. 7B: lung tissue was stained with total cells per ml BALF and flow cytometric quantification of percentage of eosinophils in BALF following final OVA challenge; *P<0.05, **P<0.01 (n=10 per group) one-way ANOVA with Tukey's post hoc test. FIG. 7C: AB-PAS, dark purple mucus-positive bronchioles (arrows); magnification ×20, scale 200 μm. FIG. 7D: Masson's trichrome; magnification ×40, scale 200 μm. FIG. 7E: hydroxyproline levels (mg/g lung tissue) in the lung. *P<0.05, **P<0.01 (n=10 per group) one-way ANOVA with Tukey's post hoc test. FIG. 7F: Western blot analysis of iNOS, arginase, MMP-9, and TIMP-1 lung tissue. Noncontiguous bands from the same Western blot are shown. FIG. 7G: ratio of TIMP-1/MMP-9 protein levels normalized to β-actin loading control. *P<0.05, (n=10 per group) one-way ANOVA with Tukey's post hoc test.

### DETAILED DESCRIPTION OF THE INVENTION

The following description sets forth numerous exemplary configurations, parameters, and the like. It should be recognised, however, that such description is not intended as a limitation on the scope of the present invention, but is instead provided as a description of exemplary embodiments.

All references, including patents and patent applications, cited in this specification are hereby incorporated by reference. No admission is made that any reference constitutes prior art. Nor does discussion of any reference constitute an admission that such reference forms part of the common general knowledge in the art, in New Zealand or in any other country.

### Definitions

In each instance herein, in descriptions, embodiments, and examples of the present invention, the terms “comprising”, “including”, etc., are to be read expansively, without limitation. Thus, unless the context clearly requires otherwise, throughout the description and the claims, the words “comprise”, “comprising”, and the like are to be construed in an inclusive sense as to opposed to an exclusive sense, that is to say in the sense of “including but not limited to”.

The term “consisting essentially of”, as used herein, may refer to the presence of a concentrate in a composition. For example, the concentrate may be at least 80% by weight of the composition, or at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.8%, or at least 99.9% by weight of the composition (% w/w). For liquids, the concentrate may be at least 80% by volume of the composition volume, or at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.8%, or at least 99.9% by volume of the composition volume (% v/v).

In the present description, the articles “a” and “an” are used to refer to one or to more than one (i.e., to at least one) of the grammatical object of the article. By way of example, “an element” can be taken to mean one element or more than one element.

Throughout this description, the term “about” is used to indicate that a value includes the standard deviation of error for the method being employed to determine the value, for example, levels of compounds or dosage levels, as described in detail herein. In particular, the term “about” encompasses a 10% to 15% deviation (positive and negative) in the stated value or range, particularly 10% deviation (positive and negative) in the stated value or range.

“Airway remodelling”, also referred to as tissue or lung remodelling, refers to the development of specific structural changes in the airway wall. Airway remodelling may include one or more of subepithelial fibrosis, myofibroblast accumulation, airway smooth muscle hyperplasia, and hypertrophy, mucous gland and goblet cell hyperplasia, and epithelial disruption. Symptoms may include decreased airway distensibility (i.e., stiffer airways), diminished elastic recoil, progressive decline in FEV1 (forced expiratory volume 1), and FVC (forced vital capacity), accelerated lung function decline, irreversible or only partially reversible airflow obstruction, dyspnea, and decreased responsiveness to respiratory therapy (e.g., asthma or COPD therapeutics).

“Asthma” refers to an inflammatory disorder of the airways of the lungs, characterized by variable and recurring breathing impairment, including airflow obstruction and bronchospasm. Airflow obstruction may be defined as reduced FEV1 and/or reduced FEV1/VC ratio. The airflow obstruction in asthma may be reversible with or without medication. Symptoms of asthma may include one or more of wheezing, coughing, chest tightness or pain, and shortness of breath. Included herein are atopic (e.g., allergen or antigen induced) and non-atopic forms of asthma, as well as exercise-induced asthma, occupational asthma, aspirin-induced asthma, and alcohol-induced asthma.

A “respiratory aid” is a composition that assists with airway function or other aspects of the respiratory system, e.g., medicines, herbal compositions, essential oils, and various compositions for inhalation.

“Airway”, “respiratory tract”, and “respiratory system” refer to any of the organs, tissues, or cellular components involved in gas exchange (i.e., breathing). This includes the upper respiratory tract, trachea, bronchi, bronchioles, alveoli, lungs, pleura and pleural cavity, and the nerves and muscles of breathing.

“Boysenberry” as used herein encompasses a Rubus hybrid berry, which includes but is not limited to a berry obtained from the plant identified as Rubus ursinus var loganobaccus cv Boysenberry, Rubus ursinus x Rubus idaeus, Rubus loganbaccus x baileyanus Britt, and Rubus idaeus x Rubus ulmifolius. Generally speaking, a Boysenberry may be derived from a cross between raspberry and blackberry plants, or between raspberry, blackberry, and loganberry plants. Included are various Boysenberry hybrids, varieties, and genetic derivatives thereof.

“Chronic obstructive pulmonary disease”, or COPD, refers to a lung disorder associated with progressive obstruction of the airways and poor airflow. Airflow obstruction may be defined as a reduction in FEV1 and/or a reduction in FEV1/VC ratio. The airflow obstruction in chronic obstructive pulmonary disease may not be fully reversible. Symptoms include but are not limited to shortness of breath, cough, and sputum production (i.e., phlegm). COPD may be associated with smoking, air pollution, poorly ventilated cooking or heating fires. A genetic component may also be involved in COPD. The disorder is also known as chronic obstructive lung disease (COLD), chronic obstructive airway disease (COAD), chronic bronchitis, pulmonary emphysema, amongst other known terminology.

“Concentrate”, for example, in relation to a Boysenberry concentrate, refers to a composition where the liquid component (e.g., juice) has been partly or mostly removed. A concentrate may be prepared from boysenberries, for example, as a puree, paste, or powder, or may be prepared from Boysenberry juice, for example, as a juice concentrate.

A “disorder” of respiratory tract includes a disease or other condition affecting any of the organs, tissues, or cellular components involved in gas exchange (i.e., breathing), as noted herein. The disorders may be an acute or chronic condition, such as inflammation and conditions that are associated with inflammation. Particular disorders of interest include asthma, chronic obstructive pulmonary disease, reactive airway disease, airway fibrosis, and airway remodeling. Other disorders are described in detail herein.

A “genetic derivative” of a Boysenberry plant refers to offspring, sports, or other cultivars that are obtained from the Boysenberry parent stock. This includes offspring obtained from a genetic cross with the Boysenberry parent, e.g., F1 progeny or F2 progeny. The term “genetic derivative” may refer to the derived plant, itself, or to its fruit.

“Fibrosis”, as in airway or pulmonary fibrosis, refers to a disruption in the regulation of collagen and other extracellular matrix components in the respiratory tract. In the airways of patients with fibrosis, there may be increased extracellular matrix deposition, such as in the reticular basement membrane region, lamina propria, and/or submucosa. Scar formation and the accumulation of excess fibrous connective tissue leads to thickening of the airway walls. Symptoms may include reduced oxygen supply, shortness of breath, chronic cough, fatigue and/or weakness, chest discomfort including chest pain, loss of appetite, and weight loss. Included are idiopathic forms of airway fibrosis, as well as airway fibrosis associated with smoking, air pollution, connective tissue disease (e.g., rheumatoid arthritis, sarcoidosis, etc), infections, medications (e.g., methotrexate, bleomycin, etc), and radiation therapy.

“Inflammation” refers to a condition characterised by one or more of: vasodilation, heat, redness, discomfort, swelling, edema, lesions, fissures, ulcerations, leukocyte extravasation, and loss of function. Included are both acute and chronic forms of inflammation, the latter of which includes inflammatory disorders, e.g., autoimmune diseases or allergic conditions. Particularly included are asthma, chronic obstructive pulmonary disease, airway fibrosis, reactive airway disease, and airway remodeling. Other inflammatory disorders are described elsewhere in this document.

As noted herein, the terms “lyophilising” and “freeze drying” are used synonymously. It will be understood that the terms “freeze drying”/“lyophilising” do not exclude the use of higher temperatures (i.e., higher than freezing temperatures). For example, higher temperatures may be used for removing residual moisture during the secondary drying phase for lyophilisation/freeze drying procedures.

A “nutraceutical” refers to a standardised composition for administration to a subject. It may be a pharmaceutical grade composition, and may maintain or improve the health of a subject, or may treat or prevent one or more disorder in a subject.

“Reactive airway disease” refers to an inflammatory airway disorder characterised by reversible airway narrowing due to external stimuli. The term can encompass other known disorders such as asthma, chronic obstructive pulmonary disease, upper respiratory tract infections, etc, or can refer to conditions that are similar to these disorders but not directly diagnosed as such, e.g., having asthma-like syndrome or asthma-like symptoms. Subjects with reactive airway disease may show one or more symptoms of coughing, wheezing, or shortness of breath upon exposure to particular stimuli, for example, smoke, vapour, fume, or other irritants.

As used herein, a “subject” may be a human or non-human animal, particularly a mammal, including cattle, sheep, goats, pigs, horses, and other livestock, including, as well, dogs, cats, and other domesticated pets. In particular aspects, the subject is a human being.

“Treating” as used herein is meant as reducing, ameliorating, or resolving a disorder, for example a respiratory disorder, such as a disease or other condition of the respiratory system. A treatment will result in the reduction, amelioration, or elimination of one or more symptoms of the disorder.

“Preventing” as used herein is meant as stopping or delaying the onset of a disorder, for example a respiratory disorder, such as a disease or other condition of the respiratory system. A preventative measure will result in the stoppage or delay of one or more symptoms of the disorder, or a lessening of symptoms if such do arise. It should be understood that the term “treating or preventing” does not exclude the possibility of obtaining both treatment and prevention (e.g., at the same time or at different times) of a disorder in any given subject. In the same way treatment of “asthma or fibrosis” does not exclude the possibility of obtaining treatment (e.g., simultaneous or not simultaneous) of both disorders.

### Boysenberries and Associated Bioactivity

The inventors have found that consumption of a Boysenberry composition reduces allergen-induced lung remodeling in a chronic model of asthma. For these experiments, the effect of Boysenberry consumption was tested on lung fibrosis, lung macrophage phenotype, and MMP-9 expression in a chronic model of allergic airway inflammation. The results demonstrated that oral Boysenberry treatment supports the development of lung macrophages that express a mixed antifibrotic, AAM (alternatively activated macrophages) phenotype with the capacity to ameliorate fibrosis and promote balanced lung repair (74; incorporated herein by reference in its entirety).

Boysenberries are known to be high in Vitamin C and fibre and contain high levels of anthocyanins (120-160 mg/100 g) that give boysenberries their deep, dark colour. The ORAC (oxygen radical absorption capacity, i.e., antioxidant level) for boysenberries is 42 μmoles/TE/g almost double that of blueberries, a well known antioxidant food. Boysenberries contain notable amounts of ellagic acid, a phenolic compound. The ellagic acid level in boysenberries is 5.98 mg/g of dry weight. Boysenberries also have a high ratio of free ellagic acid to total ellagitannins.

From the inventors' results it is evident that Boysenberries may be used in compositions for treating or preventing inflammation of the respiratory tract, treating or preventing asthma, treating or preventing chronic obstructive pulmonary disease, treating or preventing fibrosis of the respiratory tract, or treating or preventing airway remodeling. In addition, from the results shown herein, it will be understood that Boysenberry compositions may be used to restore, improve, or maintain the health of the respiratory system, for example, in one or more activities of: decreasing collagen deposition, abrogating aberrant collagen deposition, decreasing cellular infiltration into the airways, decreasing airway damage due to cellular infiltration, reducing cells in the lung fluid, e.g., inflammatory cells, reducing mucus production, reducing mucus-positive cells, decreasing hydroxyproline levels, increasing matrix metallopeptidase expression levels, e.g., protein levels, increasing MMP-9 expression levels, e.g., protein levels, increasing TGFβ expression levels, e.g., protein levels, decreasing the ratio level of TIMP-1/MMP-9, e.g., protein ratio levels, decreasing the activation or number of inflammatory cells, increasing the number or activity of alternatively activated macrophages, increasing the number or activity of arginase+ macrophages, increasing the number or activity of CD68+/CD206+/arginase+ macrophages, or decreasing iNOS expression levels, e.g., protein levels. Further uses for these composition are described in detail herein.

### Methods of Producing Boysenberry Compositions

The present invention relates generally to a composition prepared from Boysenberry. In one particular aspect, the composition is prepared from Rubus ursinus var loganobaccus cv Boysenberry. In other aspects, one or more genetic derivatives from this Boysenberry plant may be used. For example, it may be desirable to use F1 or F2 progeny from a genetic cross that includes the parent stock of the Boysenberry plant. Alternatively, any sports or other cultivars obtained from the parent may be used. It may be desirable to source the boysenberries from New Zealand, in particular, or alternatively, from Chile.

The composition is preferably prepared as a Boysenberry concentrate, for example, a Boysenberry puree, Boysenberry paste, Boysenberry powder, or Boysenberry juice concentrate. Accordingly, the composition may be prepared in liquid or powdered form, for example, a lyophilised powder, or in any other suitable dosage form. The composition may formulated as a tonic, extract, elixir, linctus, concentrate, syrup, solution, suspension, emulsion, draught, puree, paste, or as drops. In other aspects, the composition may be formulated as a gel or jelly, or a capsule, for example, with liquid or semi-liquid contents. The composition may be provided in sachet form, for example, a powder sachet, or a gel or jelly sachet. Included also are formulations comprising thin strips, or comprising solids in a capsule to mix with food or drink. Other formulas are also possible, as described herein below.

In certain aspects, it may be desirable to formulate Boysenberry (e.g., Boysenberry juice concentrate or puree) into a powder. Commercial Boysenberry powders are known and available, as noted herein. The powder may be formulated as tablets (including rapid dissolve tablets) or capsules (including extended release capsules). The tablets may be scored tablets, chewable tablets, effervescent tablets, orally disintegrating tablets, or tablets for forming a suspension. The capsules may be gel capsules, for example, and may include powdered contents. This includes gel capsules made by single piece gel encapsulation and two piece gel encapsulation. Non-gelatine capsules are also included, as well as caplets. The powder may be provided in free flowing form or as a solid cake. The composition may be provided as a powder for forming a suspension, powder for forming a solution, bulk oral granules, or bulk oral powder.

The compositions of the invention may be prepared from Boysenberry juice concentrate or puree obtained from one or more commercial sources. For example, commercial sources of New Zealand Boysenberry products include Boysenberries New Zealand Ltd, Nelson, and Tasman Bay Berries, Nelson. Commercially available products include individually quick frozen berries, Boysenberry puree, block frozen berries, Boysenberry juice concentrate, and Boysenberry powder.

The pH of the concentrate or puree may range from 3.2 to 3.8; or 3.0 to 4.0; or 3.1 to 3.9; or may be about 3.1, about 3.2, about 3.3, about 3.4, about 3.5, about 3.6, about 3.7, about 3.8, about 3.9, or about 4.0. For the Boysenberry juice concentrate, the acidity (% w/w citric acid anhydrous) may be about 1 to about 20, about 1.5 to about 15, about 2 to about 12, about 5 to about 10, about 6 to about 9, about 10, about 9, about 8.5, about 8.3, about 8.2, about 8.17, about 8.1, about 8, about 7, about 6, or about 5. In some circumstances, it may be desirable to adjust the pH of the puree or that of the final composition to approximate physiological levels. In particular, it may be useful to obtain a pH range from 6.0 to 8.0; or 6.5 to 7.5; or 6.8 to 7.2; or a pH of about 6.5, about 6.7, about 6.8, about 6.9, about 7.0, about 7.1, about 7.2, about 7.3, about 7.4, or about 7.5.

In certain aspects, the compositions of the invention may be prepared by “soft pulping” technology referred to in New Zealand Patent No. 235972 (incorporated by reference herein), which can be adapted to produce a “soft” Boysenberry puree. It may be useful to prepare the puree to have seeds removed. It may also be useful to prepare the puree with a sieve size of about 1 mm or less.

A Boysenberry juice concentrate may be prepared as a natural sugar solution that is extracted or pressed and filtered from the skin, pulp and seeds of the Boysenberry. The solution may be depectinized, filtered, and evaporated under vacuum to a specified Brix level. For example, the juice concentrate may be folded about two to about seven times the original Brix value. In particular, the concentrate may be folded about two times, about three times, about four times, about five times, about six times, or about seven times the original Brix value. Accordingly, the Boysenberry juice concentrate may have a final sugar level ranging from 55 to 75° Brix; or 59° to 69° Brix; or 61° to 66° Brix; or about 60°, about 61°, about 62°, about 63°, about 64°, about 65°, about 65.4°, about 65.5°, about 65.6°, about 66°, about 67°, about 68°, or about 69° Brix.

In particular aspects, the Boysenberry juice concentrate may be manufactured from sound, ripe graded boysenberries (e.g., Rubus ursinus var loganobaccus cv Boysenberry). The concentrate may be produced by milling, mashing and pressing into single strength juice which is centrifuged, pasteurized, depectinised, filtered and then concentrated by evaporation with aroma returned in the standardisation process. The standardised concentrate may then be packed through the hygienic filler head into the required pack style without further heat treatment. The concentrate can be checked for compliance with the definition of a pure fruit juice, for example, as defined by the FSANZ-Food Standards Australia New Zealand.

It is expected that the Boysenberry juice concentrate will be rich in colouration. For example, the Boysenberry juice concentrate may have a colour ratio (absorbance 520 nm/absorbance 430 nm) of about 1.5 to about 3.0, about 1.8 to about 2.8, about 1.9 to about 2.2, or about 1.9, about 2, about 2.01, about 2.05, about 2.1, or about 2.2. In addition, the juice concentrate may have a colour intensity (utilising Chroma meter) of about 15 to about 30, about 20 to about 28, about 21 to about 25, about 22 to about 24, or about 22, about 23, about 23.2, about 23.5, about 23.7, about 24, or about 25. The juice concentrate is also expected to be relatively clear in appearance, for example, with clarity levels of about 0.01 to about 0.1, about 0.02 to about 0.08, about 0.03 to about 0.06, about 0.04 to about 0.05, or about 0.03, about 0.04, about 0.045, about 0.047, about 0.048, about 0.05, or about 0.06. The various measurement methodologies, e.g., colour ratios, clarity, etc, are known in the art, and may be found, for example, in the AIJN code of practice in the International Fruit Juice Federation Handbook of Analysis, 1996, International Fruchtsaft-Union, Zug, Switzerland.

In initial preparatory stages, the Boysenberry fruit may undergo a pre-treatment process which may include the well known steps of ripening, inspecting, grading, and/or sorting of the Boysenberry. With regard to ripening, it is preferable to use ripe or mature Boysenberry when producing the compositions of the invention; however, rotted or decaying material is preferably avoided.

Ripeness can be assessed using widely known and used methods in the art. Ripeness can be measured prior to picking or processing the Boysenberry. In particular, ripeness may be measured using the Brix system, as noted herein. Boysenberry fruit that is overly mature or fermenting may not produce an ideal composition. Boysenberry with a Brix level below the ideal may be artificially ripened before use.

As part of the processing, the Boysenberry may be sterilised. The fruit may be passed through an assembly having one or more roller brushes for removing any adhering foreign matter. Conventional washing techniques may then be employed. For example, it is possible to use a series of spray nozzles to wash the Boysenberry. Wash additives aiding cleansing or reducing the bacteria count on the Boysenberry may be employed according to local regulations and requirements. For example, the fruit may be washed by a chlorine wash and/or an ozone impregnated water wash followed by a fresh water rinse.

The sterilized Boysenberry may then be conveyed into a hopper. This can be tapered to form a funnel to direct the boysenberries to a pressing assembly. The pressing assembly may be adapted to perform a pulping or comminution process. Such process can be relatively mild and gentle (“soft pulping”) compared to conventional fruit pulping techniques. With soft pulping, no significant disintegration or lysis of fruit cells or components. Preferably, only a minor proportion (generally less than 5-10%) of seeds is fragmented by this process.

In one embodiment, the pressing assembly performs the soft pulping of the Boysenberry by pressing the Boysenberry between a twin converging belt press. The press belts may be multiple loops rotated about a series of pulleys. The distance separating the press belts may decrease in the direction of travel of the Boysenberry. In this way, increased force may be exerted upon the Boysenberry as it travels along the length of the pressing assembly. This can produce pulping of the Boysenberry without significant damage to the seeds. This in turn prevents seeds from contaminating the pulp.

The pulp generated from the pressing assembly may be directed to a screening process, in order to separate the seeds from the pulp. In particular, the pulp may be separated from the seed using a soft mechanical screening technique. For example, a pulp finisher may be used. This includes a rotating flexible impeller which is rotated within a cone shaped screen having apertures of a predetermined size. In particular aspects, the size of the apertures is selected to permit the pulp and juice of the Boysenberry to pass through the screen while retaining a substantial portion, if not all, of the seeds within the interior cavity defined by the screen.

In certain aspects, it may be preferable to use a paste rather than a puree from the Boysenberry. A Boysenberry paste may be made as a concentrate. For example, the fruit may be heated for several hours, strained, and reduced to a thick, concentrated form. The fruit may be heated after removing the skins, or after the pulping or pureeing process. The fruit can be heated gradually, and then kept heated at a moderate temperature, with mixing. Upon thickening, the paste can be spread on a flat sheet, or transferred to a packaging, for example, a bag, tube, jar, bottle, or other container. The paste may be transferred aseptically, such that it is suitable for human consumption. It may be desired to prepare the paste from mature Boysenberry. The paste may be prepared from pulped fruit. The paste may be a smooth preparation.

The pulp (e.g., in paste or puree form) or juice concentrate may be processed by a freezing step. This may be followed by or used in conjunction with a drying step. In an alternative embodiment, the pulp is dried and processed to a powder without an intervening freezing step. For example, methods involving drum drying may be used. In the drum-drying process, a puree or paste may be dried at relatively low temperatures over rotating, high-capacity drums that produce sheets of drum-dried product. In certain aspects, an additive may be used to accelerate or otherwise assist the drying process. For example, pea starch or other drying aids may be utilised. The dried product may then be milled to a finished flake or powder form. Advantageously, drum drying techniques may be used to produce a dried composition that retains its key components, e.g., phenolic compounds, and can be easily reconstituted using liquid. For example, drum dried products may be made to be cold water soluble. As further alternatives, belt drying or convection drying may be used. Such drying methods are widely known and used in the field.

If freezing is used, it is preferable to freeze the pulp or juice concentrate as soon as possible after it is produced to maintain freshness. However, freezing may be carried out within 24 or 48 hours, as needed. Freezing methodologies are well known and need not be described in significant detail herein. Blast freezing is particularly preferred for use with the invention. The pulp or juice concentrate may be frozen in standard sized pales, which are used to collect the fresh pulp/concentrate after processing. The pulp or juice concentrate can be stored frozen (e.g., at −18° C.) until it is required.

The frozen pulp or juice concentrate may be freeze dried, i.e., lyophilised. Freeze drying techniques are widely known and commonly used. The freeze drying cycle may be about 48 hours; or ranging from 40 to 56 hours; or 12 to 36 hours; or 36 to 60 hours; or about 40 hours, about 42 hours, about 44 hours, about 46 hours, about 48 hours, about 50 hours, about 52 hours, or about 54 hours. A longer freeze drying cycle, e.g., at least 48 hours (“gentle freeze drying”), may be used to retain maximal activity. In particular aspects, the process may be carried out to such that water formation is avoided, and the moisture content is minimised during processing.

It may be desirable to use a particular lyophilisation process for obtaining the dried product. For example, a lyophilisation drying program may be used as part of an automated drying system. The lyophilisation process may include multiple drying steps, e.g., with step-wise increases and reductions in temperature. Preferably, a primary drying setting is used for sublimation, followed by one or more secondary drying settings that are used to remove residual moisture. In particular aspects, the top temperature of the lyophilisation process does not exceed 70° C. In other aspects, the temperature of the lyophilisation process ranges between −10° C. to 70° C. In one other aspect, up to 48 hours of lyophilisation is utilised.

The resulting dried product may then be milled into a powder which can then be utilised as appropriate. Milling methods are well known and widely used in the art. Standard mesh sizes may be used to produce the powder, for example, US 20, US 23, US 30, US 35, US 40, US 45, or US 50 mesh sizes may be used. The sieve size for the powder may range from 1.0 to 0.3 mm; or 0.84 to 0.4 mm; or 0.71 to 0.5 mm; or may be about 1.0 mm, about 0.84 mm, about 0.71 mm, about 0.59 mm, about 0.5 mm, about 0.47 mm, about 0.465 mm, about 0.437 mm, about 0.4 mm, about 0.355 mm, or about 0.3 mm.

To ensure minimal degradation of Boysenberry ingredients, the preparation process may be performed at a temperature of less than 40° C. In various embodiments, the process is performed at a temperature ranging from −4° C. to 40° C.; or from −1° C. to 10° C.; or from 1° C. to 6° C.; or at approximately 0° C., approximately 1° C., approximately 2° C., approximately 3° C., approximately 4° C., approximately 5° C., or approximately 6° C. These temperatures may be kept during the entire preparation process, including the storage of the whole fruit, prior to it being broken open, and during the pulping/pureeing process. For optimal results, these temperatures are kept at least from the point that the fruit has been broken open. Use of such temperatures avoids oxidation of the fruit and the use of reducing agents. In certain circumstances, it may be possible to obtain organic certification.

The processing method is preferably performed so as to prevent or at least minimise any damage or effects on the active material in the Boysenberry. To ensure optimal production methods, the resulting compositions can be monitored for activity, for example, for anthocyanin levels, polyphenol levels, and/or antioxidant activity.

Assays for polyphenols are well known in the art and are also described below. In particular, it is possible to measure gallic acid equivalents (GAE) to determine total polyphenol content. For example, the Folin-Ciocalteu method (employing the Folin-Ciocalteu reagent, also called Folin's phenol reagent or Folin-Denis reagent) may be used for colorimetric in vitro assays of phenolic compounds (75). It is expected that the total polyphenol content of a Boysenberry juice concentrate will be relatively high, for example, about 500 to about 5000 mg GAE/100 g FW, about 1000 to about 3000 mg GAE/100 g FW, about 1500 to about 2500 mg GAE/100 g FW, about 3000, about 2500, about 2000, about 1500, or about 1000 mg GAE/100 g FW. It is noted that FW indicates the fresh weight of the juice concentrate.

Anthocyanins may be quantified by HPLC. This can be used give breakdown of individual compounds and expressed as cyanidin 3-glucoside equivalents (76). For example, HPLC eluted components may be monitored at 530 nm for anthocyanins. A standard curve may be prepared using a cyanidin-3-glucoside standard (for example, from Extrasynthese) and total anthocyanins may be calculated on this basis. Other phenolics may also be analysed by HPLC, for example at 250-700 nm. A range of standards may be run, including gallic acid, ellagic acid, quercetin, rutin and catchin. Absorbance spectra and retention time of the standards may be compared with unknowns in the HPLC traces. This analysis can include measurements for ellagic acid. As non-limiting examples, the total anthocyanin content of a Boysenberry juice concentrate (expressed as cyanidin 3-glucoside equivalents) may be about 1000 to about 10,000 mg/100 g FW, about 2000 to about 8000 mg/100 g FW, about 4000 to about 7000 mg/100 g FW, about 5500 to about 6500 mg/100 g FW, or about 8000, about 7000, about 6500, about 6800, about 6000, about 5000, about 4000, or about 3000 mg/100 g FW.

Antioxidant capacity may be measured by ORAC and/or DPPH assays. The oxygen radical absorbance capacity assay is one of the most widely utilised assays to test the antioxidant potential of foods. The ORAC assay measures antioxidant inhibition of peroxyl radical-induced oxidation (77, 78, 84). Trolox, a water-soluble analogue of vitamin E, may be used as a control standard. In an additional assay, DPPH (2,2-diphenyl-1-picrylhydrazyl) may be used to show the kinetic behaviour of polyphenols as free radical scavengers. The higher the antioxidant activity, the larger the decrease of DPPH⋅ concentration. A methanolic solution of the DPPH radical changes from purple to colourless when quenched by antioxidants. The decrease in DPPH⋅ is measured at 515 nm against standard curves, e.g., Trolox and DPPH⋅ (79, 80).

As particular exemplifications, the antioxidant capacity for the Boysenberry juice concentrate may be about 10,000 to about 100,000 ORAC value (μmol Trolox/100 g FW), about 20,000 to about 80,000 ORAC value, about 30,000 to about 70,000 ORAC value, about 40,000 to about 50,000 ORAC value, or about 80,000, about 70,000, about 60,000, about 50,000, about 40,000, about 30,000, or about 20,000 ORAC value. As further exemplifications, the antioxidant capacity for the Boysenberry juice concentrate may be measured with the DPPH assay (at 100% MeOH) as about 1000 to about 6000 μmol TEAC/100 g FW, about 2000 to about 5000 μmol TEAC/100 g FW, about 2500 to about 2900 μmol TEAC/100 g FW, or about 5000, about 4000, about 3000, about 2800, about 2500, about 2000, or about 1000 μmol TEAC/100 g FW.

Alternatively or additionally, the compositions can be tested for other components, e.g., sugars, folate, and Vitamin C. The corresponding assays are widely known. For example, folate levels of the Boysenberry juice concentrate may be measured using standard methodologies (see e.g., 83), and may be about 20 μg/100 g FW, about 30 μg/100 g FW, about 40 μg/100 g FW, or about 50 μg/100 g FW, about 60 μg/100 g FW, about 70 μg/100 g FW, or about 80 μg/100 g FW, or about 10 to about 100 μg/100 g FW, about 20 to about 80 μg/100 g FW, about 30 to about 70 μg/100 g FW, about 20 to about 50 μg/100 g FW, or about 50 to about 70 μg/100 g FW.

It will be understood that other known assays may also be used to analyse the disclosed compositions (see, e.g., 85), and the invention is not limited to one particular assay for bioactive compounds, including phenolics, anthocyanins, antioxidants, vitamins, carbohydrates, etc. It will be understood also that the levels identified herein for juice concentrates can be readily extrapolated to powdered forms, as well as puree and paste forms.

In some circumstances, it may be possible to use genetic derivative of the Boysenberry plant to obtain the compositions of the invention. It is expected that a composition obtained from such derivative would share one or more of the characteristics of the compositions obtained from the original Boysenberry stock. Exemplary features include: polyphenol levels and polyphenol profiles, including anthocyanidin levels and profiles, vitamin levels, and reduction of OVA-induced inflammation, as noted above and disclosed in detail herein. Regarding the fruit itself, it is expected that the Boysenberry obtained from a genetic derivative would share a similar compositional makeup as the Boysenberry parent.

### Compositions Comprising Boysenberry

The inventors have found that Boysenberry compositions include beneficial ingredients that are useful for maintaining the health of the respiratory system, as well as treating and preventing respiratory problems. The inventors have shown that a Boysenberry concentrate is particularly efficacious for reducing airway inflammation and fibrosis. As such, the Boysenberry compositions disclosed herein can be used to support or improve overall respiratory health and/or to treat or prevent various disorders or other conditions of the respiratory tract, including inflammation, asthma, chronic obstructive pulmonary disease, airway fibrosis, and airway remodeling. In this way, the disclosed compositions are understood to be anti-inflammatory compositions, and also anti-asthmatic compositions, as well as being compositions that are active against chronic obstructive pulmonary disease, reactive airway disease, airway fibrosis, and airway remodeling.

As described herein, Boysenberry composition may comprise a juice concentrate or a powder concentrate prepared from Boysenberries. As various alternatives, the composition may consist of, or may consist essentially of, a juice concentrate or a powder concentrate prepared from Boysenberries. The Boysenberry composition may be formulated as a liquid, for example, a juice concentrate, syrup, suspension, or tonic for oral administration, or as a solution for enteral administration. Alternatively, the Boysenberry composition may be formulated as a powder to be encapsulated, tableted, or added to or incorporated in other products. Particularly encompassed are delayed release formulas, extended release formulas, as well as formulas for rapid disintegration. Capsules, for example gel capsules, are specifically encompassed, as well as sachets and chewable tablets. Additionally included are combination formulas, which include the powder of the invention mixed with other beneficial agents, e.g., one or more respiratory aids. In various aspects, the Boysenberry composition may be prepared as a nutraceutical composition, a pharmaceutical composition, a functional food or beverage, a natural ingredient (e.g., a natural additive), or a natural supplement (e.g., a dietary supplement).

It is expected that the Boysenberry composition will be prepared to include high levels of anthocyanins. For example, the composition may include about 10 to about 50,000 mg/ml total anthocyanins, or about 20 to about 40,000 mg/ml, about 25 to about 35,000 mg/ml, about 30 to about 30,000 mg/ml, about 40 to about 25,000 mg/ml, about 50 to about 20,000 mg/ml, about 60 to about 15,000 mg/ml, about 70 to about 10,000 mg/ml, about 80 to about 8000 mg/ml, about 90 to about 6000 mg/ml, about 100 to about 5000 mg/ml, about 10 to about 1000 mg/ml, about 20 to about 800 mg/ml, about 30 to about 600 mg/ml, about 50 to about 200 mg/ml, or about 50,000, about 40,000, about 35,000, about 25,000, about 20,000, about 15,000, about 12,000, about 10,000, about 8000, about 7500, about 5000, about 2500, about 2000, about 1000, about 1500, about 1200, about 1000, about 750, about 500, about 250 mg/ml, about 200 mg/ml, about 150 mg/ml, about 100 mg/ml, about 75 mg/ml, about 50 mg/ml, about 25 mg/ml, about 20 mg/ml, or about 10 mg/ml total anthocyanins, or a dry weight equivalent thereof.

In specific aspects, the Boysenberry composition may be administered at a dosage unit of about 1 mg to about 20,000 mg total anthocyanins or about 1 mg to about 2000 mg total anthocyanins, or about 5 mg to about 5000 mg, about 10 mg to about 3000 mg, about 10 to about 1000, about 15 mg to about 1500 mg, about 20 mg to about 1000 mg, about 25 mg to about 850 mg, about 30 mg to about 600 mg, about 35 mg to about 550 mg, about 50 to about 500 mg, about 5 to about 500, about 10 mg to about 200 mg, about 1 to about 400 mg, about 1 to about 200 mg, about 40 mg to about 400 mg, about 40 to about 200 mg, about 20 mg to about 80 mg, about 30 mg to about 60 mg, about 45 mg to about 55 mg, or about 20,000 mg, about 15,000 mg, about 12,000 mg, about 10,000 mg, about 7500 mg, about 5000 mg, about 4000 mg, about 3000 mg, about 2000 mg, about 1500 mg, about 1200 mg, about 1000 mg, about or about 500 mg, about 400 mg, about 300 mg, about 200 mg, about 100 mg, about 90 mg, about 95 mg, about 80 mg, about 75 mg, about 70 mg, about 65 mg, about 60 mg, about 55 mg, about 50 mg, about 45 mg, about 40 mg, about 35 mg, about 30 mg, about 25 mg, about 20 mg, or about 10 mg total anthocyanins. In particular aspects, the dosage unit may be about 50 mg to about 500 mg total anthocyanins. The dosage units as noted above may be administered once per day, twice per day, or three times per day, or more as needed. An exemplary, and non-limiting, daily dosage may be about 10 mg to about 1000 mg total anthocyanins. The dosage may be adjusted for pediatric, geriatric, overweight, underweight, or other patients, where required.

If a Boysenberry juice concentrate is made by standard commercial production methods (large or small scale), or obtained from commercial sources, the juice concentrate may be administered at a dosage unit of about 0.5 to about 50 ml, about 0.5 to about 20 ml, about 0.5 to about 10 ml, about 1 to about 9 ml, about 2 to about 8 ml, about 3 to about 7 ml, about 4 to about 6 ml, or about 50, about 40, about 30, about 20, about 10, about 9, about 8, about 7, about 6, about 5, about 4, about 3, about 2, about 1, or about 0.5 ml of Boysenberry juice concentrate. In particular aspects, the dosage unit may be about 5 ml of Boysenberry juice concentrate. The various dosage units may be administered once per day, twice per day, or three times per day, or more as needed. Dosage modification can be made for patient size and age in accordance with known methods.

In certain circumstances, it may be desirable to isolate or enrich the polyphenols from the Boysenberry. In particular, it may be advantageous to use the Boysenberry to obtain polyphenol enriched compositions, phenolic concentrates, or compositions comprising isolated phenolics, e.g., isolated anthocyanins. For example, the compositions of the invention may be enriched for polyphenols such that their concentration is increased relative to the other components of the Boysenberry, e.g., sugars. In particular aspects, the compositions of the invention may include polyphenols that have been isolated away from (e.g., purified from) the other components of the Boysenberry. Methods of enriching and extracting polyphenols are widely known in the art (e.g., 81, 82). The resulting composition may include at least 2 times, at least 3 times, at least 4 times, at least 5 times, or at least 10 times the amount of polyphenols compared to the composition prepared without polyphenol enrichment or isolation steps. The polyphenol enriched compositions, phenolic concentrates, and compositions comprising isolated phenolics may be dried as a powder, and used in accordance with the present invention.

The dosage form may contain excipients, for example, one or more anti-adherents, binders, coatings, disintegrants, flavours, colours, sweeteners, lubricants, glidants, flow agents, anti-caking agents, sorbents, or preservatives. Useful excipients include but are not limited to: stearin, magnesium stearate, and stearic acid; saccharides and their derivatives, e.g., disaccharides: sucrose, lactose; polysaccharides and their derivatives, e.g., starches, cellulose or modified cellulose such as microcrystalline cellulose and cellulose ethers such as hydroxypropyl cellulose; sugar alcohols such as isomalt, xylitol, sorbitol and maltitol; proteins such as gelatin; synthetic polymers such as polyvinylpyrrolidone, polyethylene glycol; fatty acids, waxes, shellac, plastics, and plant fibres, e.g., corn protein zein; hydroxypropyl methylcellulose; crosslinked polymers, e.g., crosslinked polyvinylpyrrolidone (crospovidone), and crosslinked sodium carboxymethyl cellulose (croscarmellose sodium); sodium starch glycolate; silicon dioxide, fumed silica, talc, and magnesium carbonate.

It is expected that the Boysenberry compositions disclosed herein will include various components, for example, carbohydrates and polyphenols, and in particular, anthocyanidins. Particular anthocyanidins of interest include cyanidins and rutinosides, such as cyanidin-3-O-sophoroside, cyanidin-3-O-glucoside, epicatechin, cyanidin-3-O-glucosylrutinoside, cyanidin-3-O-rutinoside, cyanidin-3-(6′-p-coumaryl)glycoside-5-glycoside, cyanidin-3-O-glycoside, cyanidin-3,5-diglycoside, and cyanidin-3-O-2G-glucosylrutinoside. The Boysenberry compositions of the invention may also include various carbohydrates, and in particular, various sugars, including neutral sugars. As to neutral sugars, the compositions in the invention may include one or more of: fructose and glucose.

### Methods of Using Boysenberry Compositions

As noted above, the Boysenberry compositions disclosed herein can be used to support or improve overall respiratory health and/or to treat or prevent various conditions of the respiratory tract, including inflammation, and respiratory disorders associated with inflammation, such as asthma, chronic obstructive pulmonary disease, reactive airway disease, airway fibrosis, and airway remodeling. Other conditions associated with inflammation in the respiratory tract include: allergy or allergic disorders, emphysema, bronchitis, respiratory bronchiolitis, interstitial lung disease, inflammatory airway disease, fibrosing alveolitis, intrinsic alveolitis, pulmonary eosinophilia, pulmonary vasculitis, pneumonia, interstitial pneumonia, desquamative interstitial pneumonia, lymphoid interstitial pneumonia, nonspecific interstitial pneumonia, eosinophilic pneumonia, pneumonitis, pleurisy (pleuritus), pleural effusion, cystic fibrosis, primary ciliary dyskinesia, acute respiratory distress syndrome (ARDS), sarcoidosis, dermatomyositis, toxocariasis, Wegener's granulomatosis, Langerhans cell histiocytosis, Sjogren's syndrome, Kartagener syndrome, vocal cord dysfunction, spasmodic croup, autoimmune disease such as lupus, reflexive vasomotor disease, and autonomic disorders. Additional factors associated with inflammation in the respiratory tract include smoking, air pollution, allergens, infection (e.g., viral or bacterial), certain medication (e.g., chemotherapeutic agents), radiation treatment, medical devices (e.g., ventilators), and surgery.

The Boysenberry compositions of the invention find use for treating or preventing respiratory tract inflammation, asthma, chronic obstructive pulmonary disease, airway fibrosis, airway remodeling, or other conditions described herein. As exemplary dosages, the compositions may be administered at dosages to obtain about 0.1 to about 200 mg/kg, about 0.2 to about 180 mg/kg, about 0.25 to about 150 mg/kg, about 0.5 to about 125 mg/kg, about 0.6 to about 100 mg/kg, about 0.7 to about 90 mg/kg, about 0.1 to about 50 mg/kg, about 0.1 to about 20 kg/mg, about 0.1 to about 10 mg/kg, about 0.1 to about 5 mg/kg, about 0.1 to about 1 mg/kg, about 1 to about 20 mg/kg, about 1 to about 10 mg/kg, 1 to about 5 mg/kg, or about 200 mg/kg, about 100 mg/kg, about 90 mg/kg, about 80 mg/kg, about 70 mg/kg, about 60 mg/kg, about 50 mg/kg, about 40 mg/kg, about 30 mg/kg, about 20 mg/kg, about 10 mg/kg, about 9 mg/kg, about 8 mg/kg, about 7 mg/kg, about 6 mg/kg, about 5 mg/kg, about 4 mg/kg, about 3 mg/kg, about 2 mg/kg, about 1 mg/kg, about 0.9 mg/kg, about 0.8 mg/kg, about 0.7 mg/kg, about 0.6 mg/kg, about 0.5 mg/kg, about 0.4 mg/kg, about 0.3 mg/kg, about 0.2 mg/kg, or about 0.1 mg/kg, of total anthocyanins in relation to patient body weight. In particular aspects, the dosage may be about 0.1 mg/kg to about 10 mg/kg. The dosages as indicated above may be administered once per day, twice per day, three times per day, or more, as needed. Administration may be made with food, or before a meal. The appropriate dosage and dosage form will be readily determined by a person of skill in the art.

Various routes of administration may be used for the Boysenberry compositions, including enteral administration and oral administration. Oral administration may be by tablet, capsule, sachet, drops, elixir, linctus, solution, emulsion, suspension, draught, puree, paste, syrup, gel, jelly, tonic, or other known means. Enteral administration may be by duodenal tubing or gastric tubing, including nasogastric tubing. Different means of administration are known in the art and may be utilised by a skilled person. The compositions disclosed herein are not limited to a particular form for administration.

It may be useful to add one or more phenolic compounds to the compositions of the invention, to further supplement the phenolic activity therein. Exemplary compounds include but are not limited to: phenolic derivatives such as phenolic acid, and flavonoids such as lignins, proanthocyanidins, anthocyanins, anthocyanidins, isoflavones, catechins, tannins, quercetin, naringenin, and hesperidin. Specific anthocyanin compounds of interest are described herein. Particularly encompassed are phenolic compounds extracted from one or more of: tea, cocoa, wine, soybeans, feijoa, citrus fruits, apples, grapes, berries, and kiwifruit. Specific phenolics include but are not limited to: ellagic acid, chlorogenic acid, catechin, epicatechin, kaemferol, E-caffeoyl-3-glucoside, E-caffeoyl-4-glucoside, neochlorogenic acid, phlorizin, procyanidin B1 and B2, quercetin, qurecetin rhamnoside, and querecetin rutinoside.

As additional aspects, the compositions of the invention may be co-administered with one or more respiratory aids. A respiratory aid may be a medication, prescription or non-prescription, or an alternative treatment, such as a herbal remedy, or an essential oil, e.g., for vaporisation and/or inhalation. Of particular interest is use of the Boysenberry composition of the invention as a respiratory treatment during and/or following other respiratory treatments. For example, the Boysenberry composition may be formulated as a combined dosage form with one or more medicines or alternative treatments. Alternatively, the Boysenberry composition may be administered as a separate dosage form along with one or more medications or alternative treatments. A respiratory aid may have one or more physiological effects, for example, anti-inflammatory, anti-spasmodic, bronchodilation, and/or muscle relaxation effects. Any respiratory aid may be long or short acting, and may be directed to a particular disorder, such as asthma, chronic obstructive pulmonary disease, etc.

Exemplary medications include but are not limited to bronchodilators, including short-acting bronchodilators such as albuterol (e.g., Vospire ER), levalbuterol (e.g., Xopenex), ipratropium (e.g., Atrovent), albuterol/ipratropium (e.g., Combivent), corticosteroids such as fluticasone (e.g., Flovent, Flovent Diskus, Flovent HFA), budesonide (e.g., Pulmicort, Pulmicort Flexhaler), mometasone (e.g., Asmanex), beclomethasone (e.g., QVAR), flunisolide (e.g., Aerospan), prednisolone, methylprednisolone, and hydrocortisone, methylxanthines such as theophylline (e.g., Theochron, Theo-24, Elixophyllin), long-acting bronchodilators such as aclidinium (e.g., Tudorza), arformoterol (e.g., Brovana), formoterol (e.g., Foradil, Perforomist), glycopyrrolate (e.g., Seebri Neohaler), indacaterol (e.g., Arcapta), olodaterol (e.g., Striverdi Respimat), salmeterol (e.g., Serevent), tiotropium (e.g., Spiriva), and umeclidinium (e.g., Incruse Ellipta), combinations of two or more long-acting bronchodilators such as glycopyrrolate/formoterol (e.g., Bevespi Aerosphere), glycopyrrolate/indacaterol (e.g., Utibron Neohaler), tiotropium/olodaterol (e.g., Stiolto Respimat), umeclidinium/vilanterol (e.g., Anoro Ellipta),

Further exemplary medications include but are not limited to combinations of inhaled corticosteroid(s) and long-acting bronchodilator(s) such as budesonide/formoterol (e.g., Symbicort), fluticasone/salmeterol (e.g., Advair, Advair Diskus), and fluticasone/vilanterol (e.g., Breo Ellipta), phosphodiesterase-4 inhibitors such as roflumilast (e.g., Daliresp), beta agonists, including short-acting beta agonists such as albuterol (e.g., ProAir HFA, Ventolin HFA), and levalbuterol (e.g., Xopenex HFA), anticholinergics such as ipratropium bromide (e.g., Atrovent HFA), long-acting beta antagonists (LABAs) such as formoterol (Perforomist), and salmeterol (e.g., Serevent Diskus), leukotriene modifiers such as montelukast (Singulair), zafirlukast (Accolate), and zileuton (e.g., Zyflo, Zyflo CR), immunomodulators such as mepolizumab (Nucala), omalizumab (e.g., Xolair), reslizumab (e.g., Cinqair), bronchodilators such as epinephrine (e.g., Primatene Mist, Bronkaid, Asthmahaler), ephedrine, and theophylline-ephedrine (e.g., Primatene tablets).

### EXAMPLES

The examples described herein are provided for the purpose of illustrating specific embodiments of the invention and are not intended to limit the invention in any way.

### Example 1

### Overview

Lung fibrosis negatively impacts on lung function in chronic asthma and is linked to the development of profibrotic macrophage phenotypes. Epidemiological studies have found that lung function benefits from increased consumption of fruit high in polyphenols. However, previous studies have not investigated Boysenberry compositions, or effects on fibrotic or remodeling in airway systems.

The inventors investigated the effect of Boysenberry consumption, in both therapeutic and prophylactic treatment strategies in a mouse model of chronic antigen-induced airway inflammation. Boysenberry consumption reduced collagen deposition and ameliorated tissue remodeling alongside an increase in the presence of CD68+CD206+arginase alternatively activated macrophages in the lung tissue. The decrease in tissue remodeling was associated with increased expression of profibrolytic matrix metalloproteinase-9 protein in total lung tissue.

The inventors identified alternatively activated macrophages in the mice that consumed Boysenberry as a source of the matrix metalloproteinase-9. The inventors hypothesise that oral Boysenberry treatment moderate chronic tissue remodeling by supporting the development of profibrolytic alternatively activated macrophages expressing matrix metalloproteinase-9. Regular Boysenberry consumption therefore has the ability to moderate chronic lung remodeling and fibrosis in asthma and other chronic pulmonary diseases.

### Example 2

### Materials

Anti-actin (clone AC-15), ovalbumin (OVA), 4% formalin, Tween 20, trans-hydroxyproline, 3,3′-diaminobenzidine (DAB) substrate, ketamine/xylazine, and all other chemicals were obtained from Sigma (Auckland, NZ). Alum was obtained from Serya (Heidelberg, Germany). The Boysenberry juice was obtained as New Zealand 65 Brix Boysenberry juice concentrate kindly provided by Berryfruit Export NZ, currently trading as Boysenberries New Zealand Ltd (Nelson, New Zealand). The 65 Brix Boysenberry juice concentrate from Berryfruit Export NZ was diluted in sterile water to obtain a concentrate of 100 mg/ml total anthocyanins. From this, a further dilution was prepared to obtain a dosage of 10 mg/kg of total anthocyanins. This further dilution is noted as Boysenberry solution.

Anti-mouse polyclonal inducible nitric oxide synthase (iNOS) (ab3523), arginase, TIMP-1 (ab38978), and MMP-9 (ab38898) were obtained from Abcam (Cambridge, UK). Antibodies against mouse CD68 (clone FA-11) CD3e, CD8a, CD4, CD11b, CD11c, and Gr-1 were obtained from BioLegend (San Diego, Calif.) and anti-CD206 (clone MR5D3) was obtained from AbDSerotec (Oxford, UK). Anti-mouse SiglecF, MHCII, and CD45 were from BD Biosciences (San Jose, Calif.).

TGFβ ELISA kit was obtained from R&D Systems (Minneapolis, Minn.). Vectastain Elite ABC staining kit was from Vector Laboratories (Burlingame, Calif.). Bio-Plex multiplex cytokine assays for IL-4, IL-5, IL-6, IL-13, and IFNγ, DC Lowry protein assay kit, and PVDF membrane were from Bio-Rad (Hercules, Calif.). BSA, NuPage 4-12% gels, MES running buffer, sample loading buffer, Novex sharp prestained, and MagicMark XP protein standards and all other buffers were from Life Technologies (Auckland, NZ).

### Example 3

### Animals

C57BL/6J male mice were bred and group housed (5 per cage) in conventional polycarbonate cages with a filter top, in a specific pathogen-free animal facility at the Malaghan Institute of Medical Research, Wellington, New Zealand. All experimental procedures were approved by the Victoria University of Wellington Animal Ethics Committee (approval number 2011R3M).

Mice were maintained on a 12-h light-dark cycle, at 21±2° C. ambient temperature with freely available irradiated standard laboratory rodent chow (Specialty Feeds, Glen Forrest, WA, Australia) and acidified water.

### Example 4

### OVA-Induced Airway Inflammation and Oral Boysenberry Treatment

Six-week-old mice were randomized into experimental groups (n=10 per group) and primed intraperitoneally (ip) with 100 μg OVA in 200 μl alum adjuvant on day 0. On day +7 mice were challenged intranasally (i.n.) with 100 μg OVA or PBS.

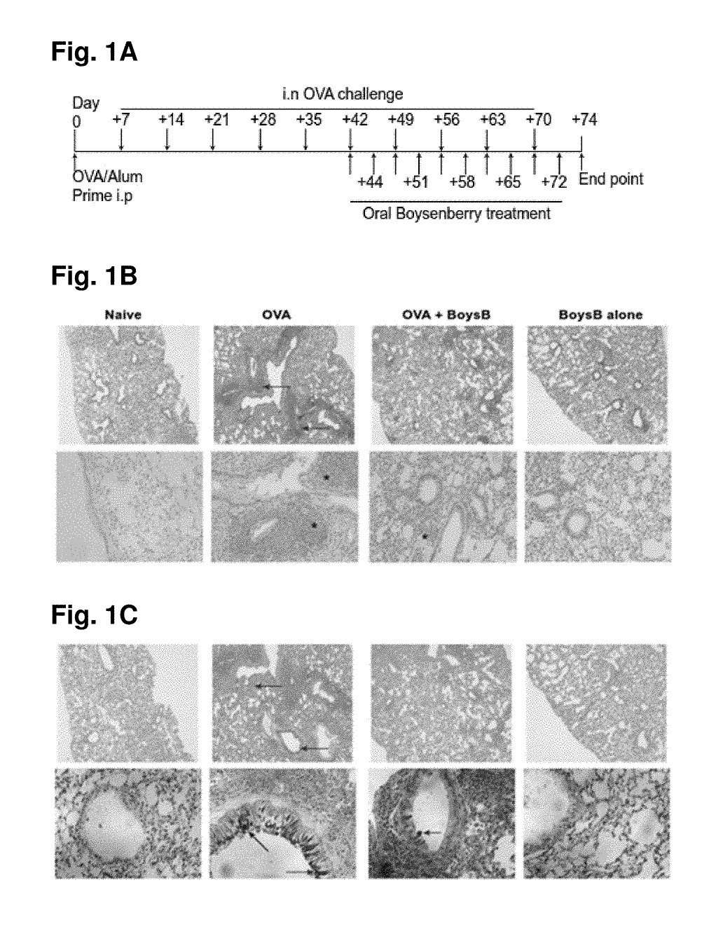

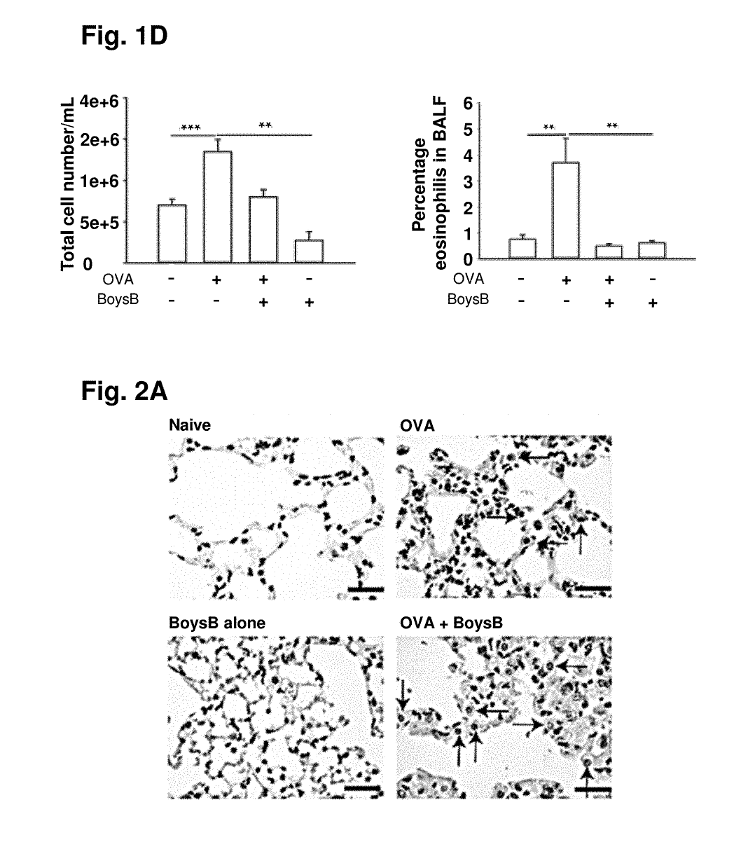

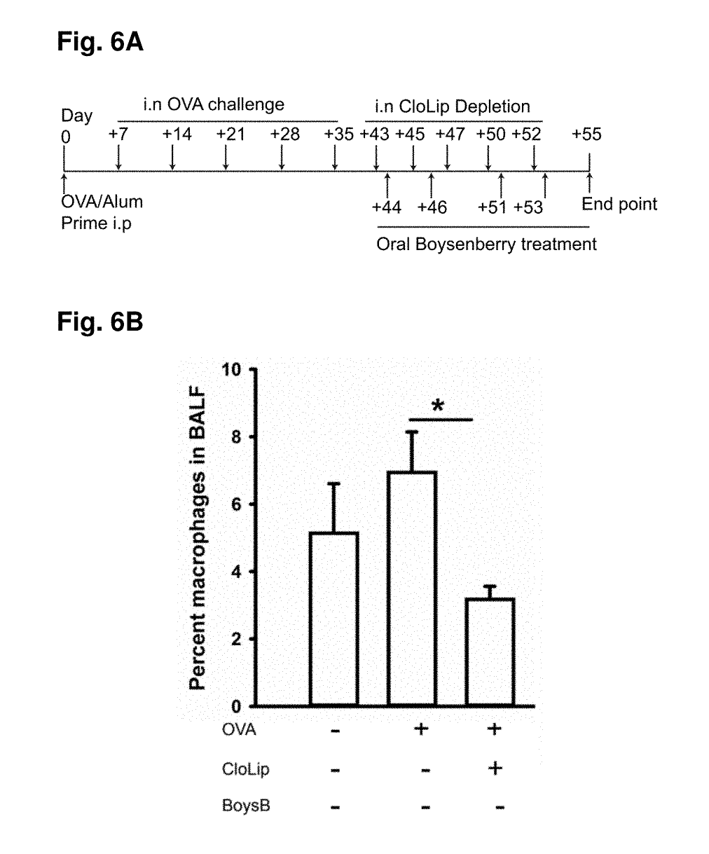

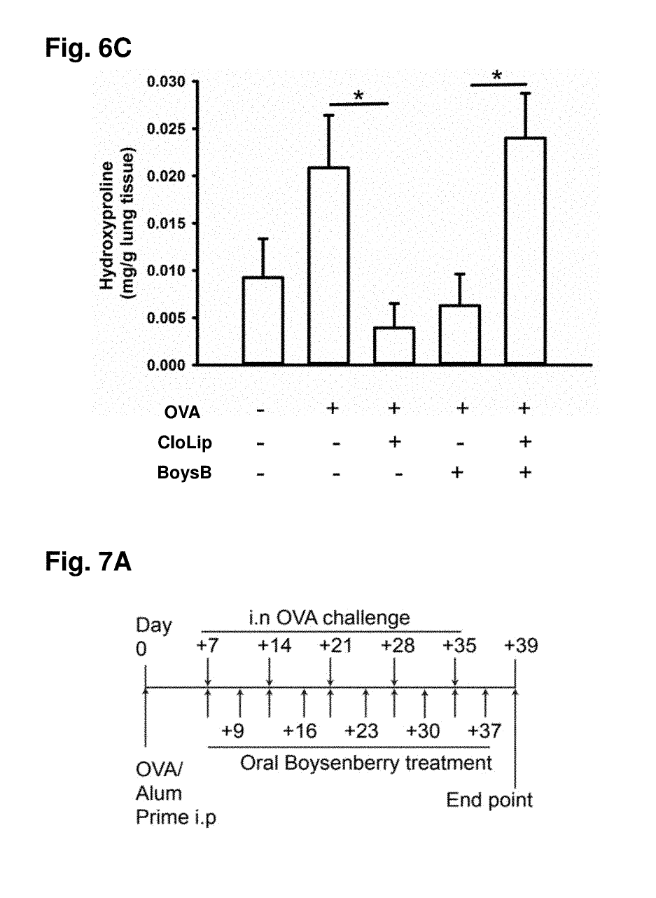

To establish chronic disease the i.n. challenge was repeated weekly (FIGS. 1A and 6A). Four days following the last i.n. OVA challenge mice were euthanized (ketamine/xylazine overdose) and bronchial-alveolar lavage fluid (BALF), serum, mediastinal lymph nodes and lung tissue were collected.

For the treatment studies mice were fasted overnight before being orally gavaged with 250 μl of Boysenberry solution (see above); dosage at 10 mg/kg of total anthocyanins) or sterile water on the day of OVA challenge and again 2 days post-OVA challenge (FIGS. 1A and 6A).

### Example 5

### Clodronate Liposome Depletion of Lung Macrophages and Tissue Analysis

Clodronate liposomes were prepared as previously described (58). Chronic OVA-induced tissue damage was established over 5 wk. Mice were then treated intranasally with 100 μl clodronate liposomes the day prior to each oral gavage with 250 μl of Boysenberry solution (see above; dosage at 10 mg/kg of total anthocyanins) or sterile water (FIG. 6A). Two days following the last oral gavage mice were euthanized (ketamine/xylazine overdose) and BALF, serum, mediastinal lymph nodes, and lung tissue were collected.

Cells isolated from the BALF were stained for key surface markers to identify monocytes/macrophages (CD45+/CD11b+/Cd11c+/MHCIIlow) and eosinophils (CD45+/CD11b+/siglecF+) by flow cytometry as previously described (52). TGFβ ELISA and Bio-Plex multiplex cytokine assays were performed on lung tissue supernatants following the manufacturer's instructions. Lung tissue was fixed in 4% formalin, sectioned, and stained with hematoxylin and eosin (H&E), Masson's Trichrome or Alcian blue-periodic acid-Schiff (AB-PAS) stains (Dept. of Pathology, Wellington School of Medicine, University of Otago, Wellington, NZ).

Further sections were cut for immunological labeling. Lung sections were incubated with biotin-conjugated MMP-9, then labelled with DAB and counter-stained with hematoxylin. Other tissue sections were incubated with fluorescently labelled CD68 (31), CD206 (57), and arginase or MMP-9 (44), then counterlabelled with DAPI-containing mounting medium.

All sections were imaged on an Olympus BX51 compound microscope and captured by using cellSens (Olympus NZ) software, bright light in colour and fluorescence in grayscale. Fluorescence images were processed (cropped, false coloured, and merged) in Pixelmator image software (Vilnius, Lithuania). Fluorescently labelled cells were quantified by four independent, blinded observers. Cells were counted in random fields from multiple animals and scored as negative, single positive, or double positive for CD68, CD206, arginase, or MMP-9. Data were expressed as a percentage of total cells counted.

### Example 6

### Biochemical and Molecular Biological Tissue Analysis and Statistical Analysis

Biochemical and molecular biological tissue analysis. Lung tissue was snap frozen and stored at −70° C. Lung collagen was quantified by the hydroxyproline assay as previously described (2). For Western blotting, tissue was homogenized in protein lysis buffer (Tris-HCl, NaCl, 10% Nonidet P-40, 10% sodium deoxycholate, 100 nM ETDA, pH 7.4 with protease and phosphatase inhibitors). Protein concentration was quantified by a Lowry protein assay as per the manufacturer's instruction.

Samples (30 μg protein) were separated by SDS-PAGE gel electrophoresis under reducing conditions and transferred onto PVDF membrane. Nonspecific protein binding was blocked with 3% BSA (10 mM PBS with 0.2% Tween 20) and the membranes were probed overnight with primary antibodies specific to iNOS (64), arginase (53), MMP-9 (44), and TIMP-1 (55), or β-actin (12) loading control (4° C.). Membranes were washed and incubated with horseradish peroxidase-conjugated secondary antibodies and visualized by chemiluminescence on a Carestream Gel Logic Pro 6000 imager. Protein expression was densitometrically quantified and normalized to β-actin with Imagers Gel analysis tool (50). Images were processed and cropped in Pixelmator image software.

Data were analysed by one-tailed Student's t-test for comparisons between two groups or one-way ANOVA with Tukey's post hoc test for comparisons between three or more groups as indicated (Prism, GraphPad, San Diego, Calif.). P<0.05 or less was considered statistically significant.

### Example 7

### Results—Boysenberry Consumption Ameliorates OVA-Induced Airway Inflammation

To investigate the effect of Boysenberry treatment on established lung remodeling, mice were challenged weekly with intranasal OVA for 5 wk, then challenged weekly with OVA for an additional 5 wk alongside weekly oral treatment with Boysenberry (FIG. 1A).

As shown in FIG. 1B, lung tissue from OVA-challenged mice exhibited increased cellular infiltrate and loss of lung structure. OVA-induced cellular infiltrate and lung damage were decreased in Boysenberry-treated mice (FIG. 1B). Staining of lung tissue for mucus production identified fewer mucus-positive cells in OVA-challenged mice receiving Boysenberry treatment compared with OVA only-challenged mice (FIG. 1C). Boysenberry treatment alone had no effect on cellular infiltration, lung structure, or mucus production.

### Example 8

### Results—Boysenberry Treatment Increases AAMs in the Lung of OVA-Challenged Mice

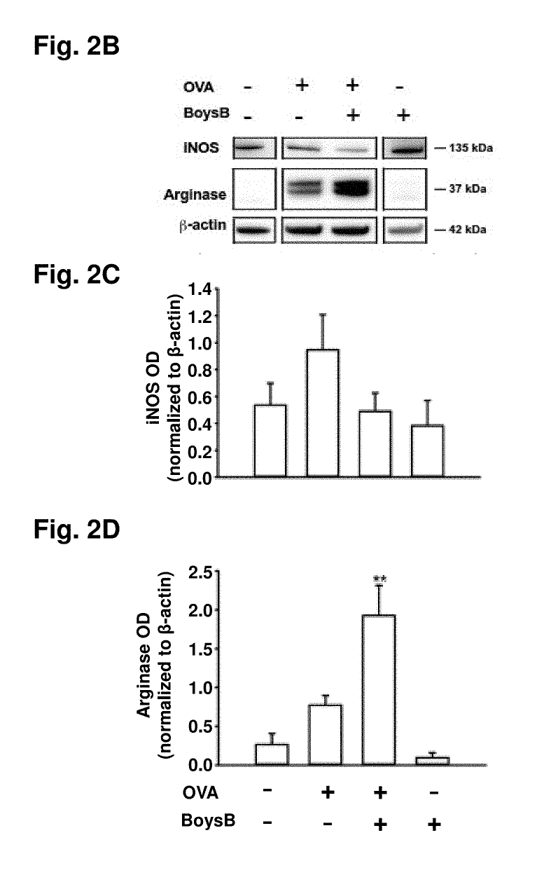

H&E-stained lung tissue sections showed more macrophages in OVA/Boysenberry-treated mice compared with OVA mice (FIG. 2A). Immunoblot analysis of lung tissue identified a decrease in iNOS expression in the lung tissue of OVA/Boysenberry-treated mice compared with OVA challenge alone (FIG. 2B and FIG. 2C). At the same time, an increase was observed in arginase expression in OVA-challenged mice (FIG. 2B and FIG. 2D). that was further enhanced in OVA-challenged mice treated with Boysenberry. Arginase expression was not affected by Boysenberry treatment alone.

AAMs expressing arginase are closely associated with lung remodeling (29). To determine whether the observed lung macrophages were alternatively activated, lung tissue was stained with fluorescently labelled antibodies for the macrophage marker CD68 and the AAM markers CD20 and arginase.

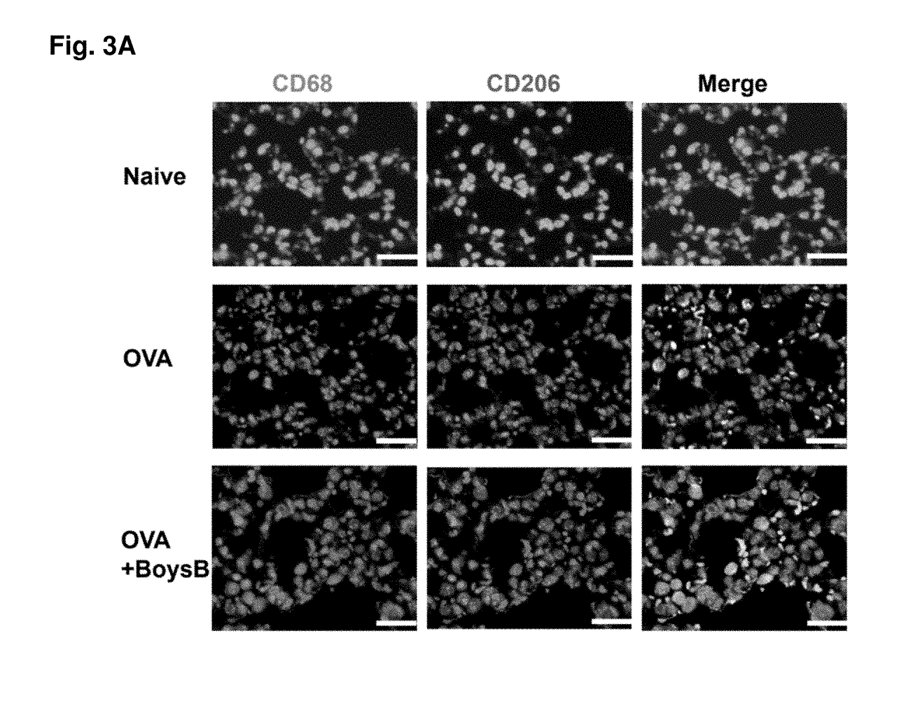

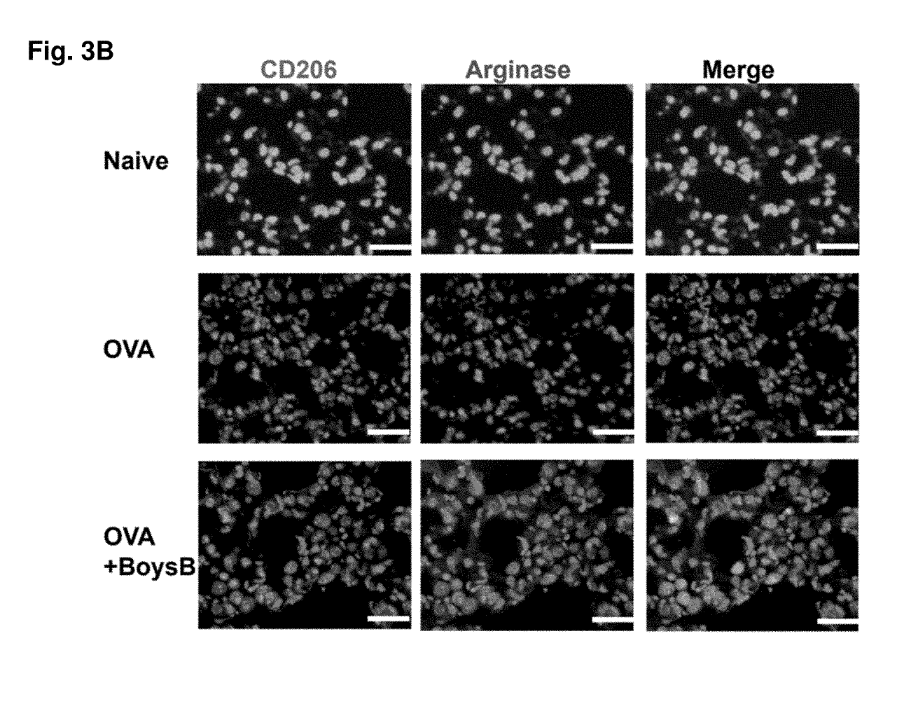

Lung tissue from OVA/Boysenberry-treated mice showed an increase in CD68+CD206+arginase+ macrophages compared with OVA-challenged mice (FIG. 3). Quantitative analysis of the CD68+CD206+arginase+ macrophages further confirmed a significant increase in the percentage of CD68+CD206+arginase+ macrophages in the lung tissue of OVA/Boysenberry-treated mice compared with OVA-challenged mice (60.00±3.54% compared with 23.47±5.61%, P<0.001, one-tailed Student's t-test). Together these data identify an increase in the number of lung macrophages expressing an alternatively activated phenotype in OVA-challenged mice receiving Boysenberry treatment.

### Example 9

### Results—Boysenberry Treatment Decreases OVA-Induced Collagen Deposition and Increases MMP-9 Expression in the Lung

Increased AAMs and arginase expression are commonly associated with tissue fibrosis (14, 27, 66); therefore the effect of Boysenberry treatment was investigated for OVA-induced collagen deposition in the lung. Following this the levels of hydroxyproline were measured in the lung tissue as a surrogate marker of collagen deposition (2, 63).

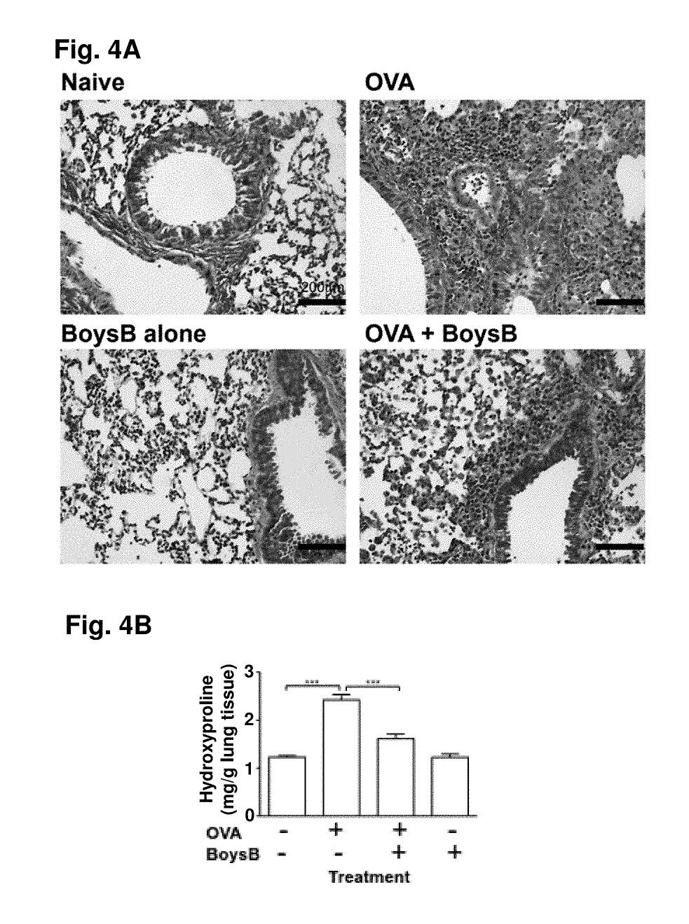

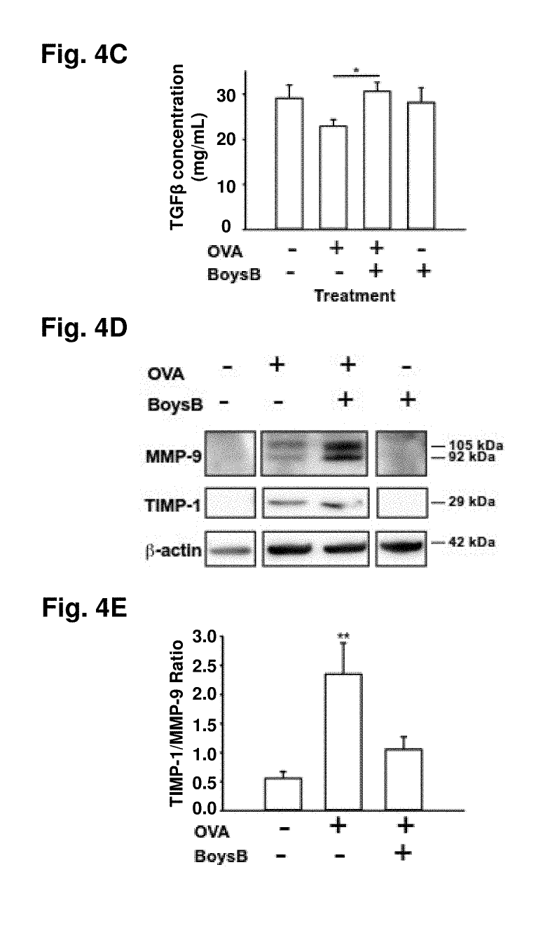

OVA challenge alone resulted in abnormal collagen deposition in the airways with signs of collagen invasion throughout the lung tissue that was abrogated in the lungs of OVA/Boysenberry-treated mice (FIG. 4A). In addition, there was a significant drop in the levels of hydroxyproline in the lungs of OVA-challenged mice treated with Boysenberry, confirming that Boysenberry treatment ameliorated OVA-induced collagen deposition (FIG. 4B). Boysenberry restored the OVA-induced decrease in the levels of TGFβ in the lung (FIG. 4C) but did not affect the levels of IL-4, IL-5, IL-6, IL-13, or IFNγ (data not shown).

To determine how Boysenberry treatment could be moderating lung fibrosis the expression of MMP-9 was measured in the lung tissue by immunoblot.

It was determined that MMP-9 expression was increased in OVA-challenged mice treated with Boysenberry compared with mice challenged with OVA alone (FIG. 4D). Boysenberry treatment alone did not affect MMP-9 levels in the lung. Tissue inhibitor of matrix metalloproteinases-1 (TIMP-1) is the endogenous inhibitor of MMP-9 (49). The ratio of TIMP-1/MMP-9 expression significantly increased in the lung tissue of chronic OVA-challenged mice and this increase was reversed with Boysenberry treatment (FIG. 4E). These results indicate that Boysenberry-mediated reduction in collagen deposition and tissue remodeling was associated with elevated production of fibrolytic MMP-9 and a subsequent rebalance in the ratio of TIMP-1/MMP-9.

### Example 10

### Results—Alternatively Activated Macrophages are a Source of MMP-9 Protein in the Lungs of OVA/Boysenberry-Treated Mice

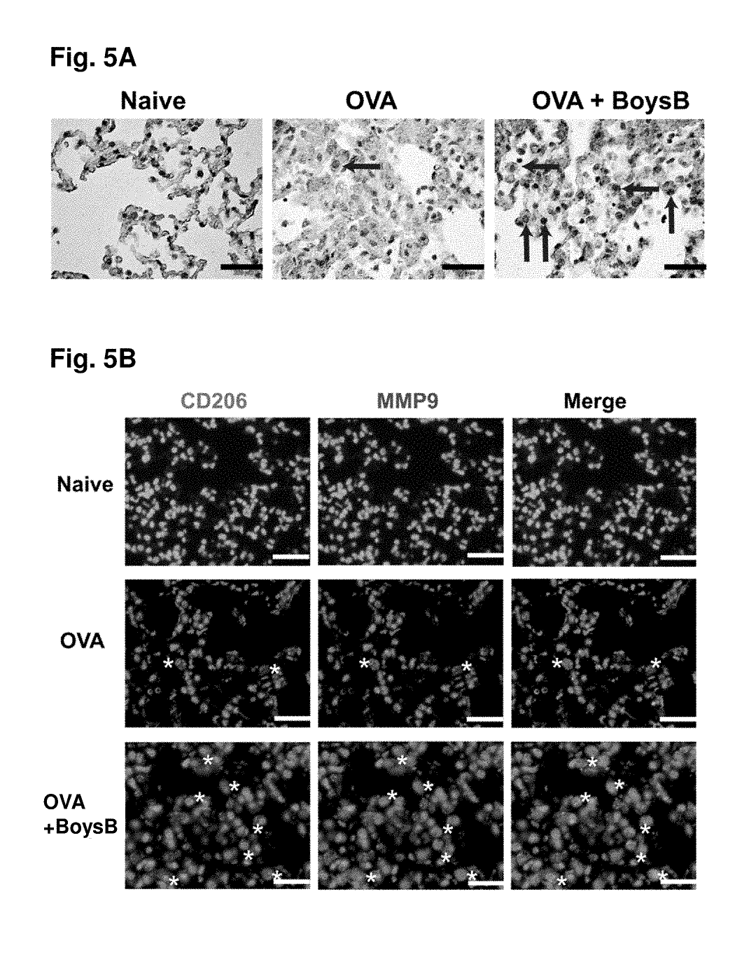

Lung tissue slides were analysed to identify potential cellular sources of MMP-9. DAB-MMP-9 staining identified a high degree of MMP+ cells exhibiting macrophage morphology in OVA/Boysenberry-treated mice compared with OVA-treated controls (FIG. 5A). Immunofluorescent staining (FIG. 5B) and quantitative analysis of the lung tissue confirmed that there were more MMP-9+/CD206+/CD68+ cells present in OVA/Boysenberry-treated lungs than those challenged with OVA alone (39.30±6.39 vs. 21.07 35 5.82%; P<0.05, one-tailed Student's t-test). These results identify CD206+/CD68+ AAMs as a source of the increased MMP-9 protein levels.

### Example 11

### Results—Depletion of Lung Macrophages Reduces the Beneficial Effect of Boysenberry Consumption on Tissue Remodeling in Established Chronic Lung Inflammation

Next, the inventors looked at the effect of depleting lung macrophages on the beneficial effects of Boysenberry on chronic lung inflammation. Macrophages were depleted by administration of clodronate liposomes after establishing chronic lung inflammation and remodeling, and prior to administration of each Boysenberry treatment (FIG. 6A). It was confirmed that significant depletion of the lung macrophages had been obtained by flow cytometry (FIG. 6B) and that this was associated with a significant reduction in hydroxyproline levels in the lung of OVA-challenged mice treated with Boysenberry (FIG. 6C). These data indicate that Boysenberry requires macrophages to mediate its beneficial effects on lung tissue remodeling.

### Example 12

### Results—Boysenberry Treatment Prophylactically Prevents OVA-Induced Airway Inflammation

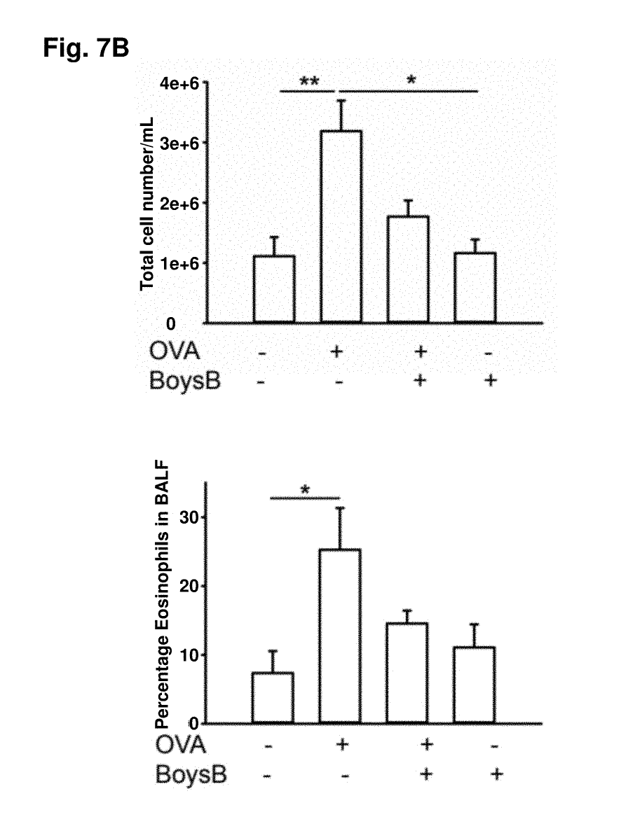

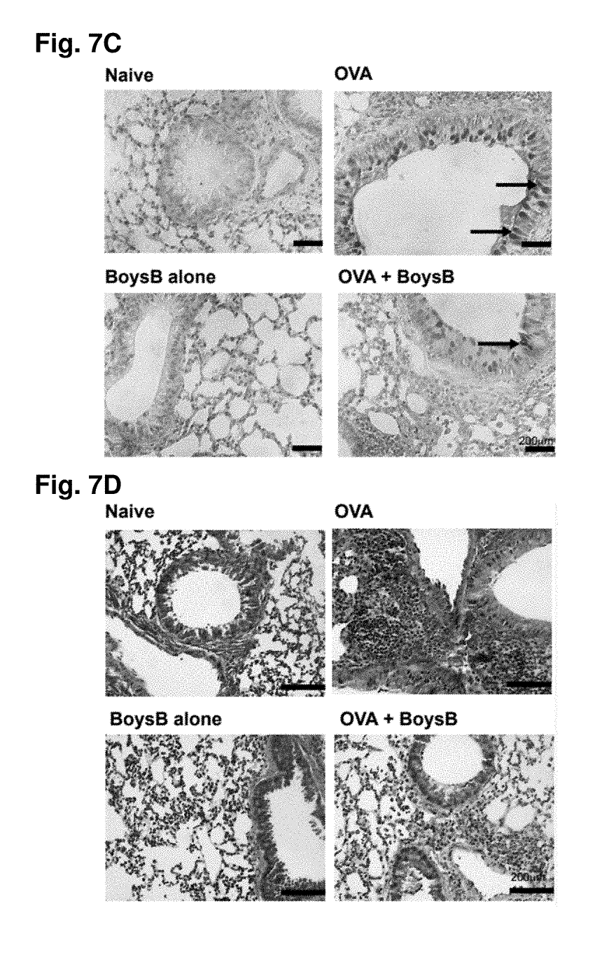

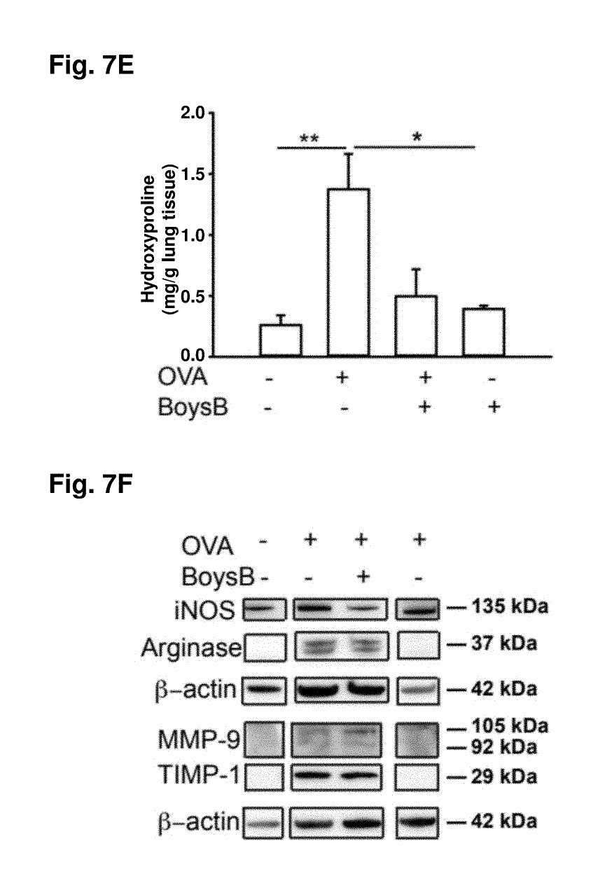

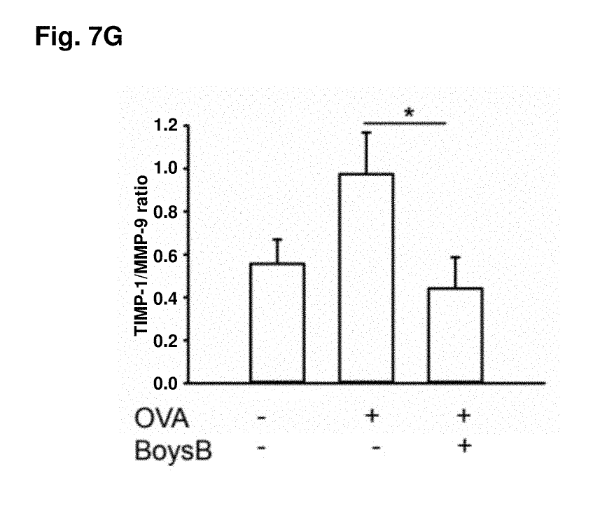

Finally, the effect of Boysenberry treatment was tested using a prophylactic dosing regimen (FIG. 7A). Again, Boysenberry treatment resulted in abrogation of OVA-induced tissue remodeling and significantly reduced cells in the lung lavage fluid (FIG. 7B-FIG. 7D). This was associated with lower levels of hydroxyproline in the lung tissue and a decrease in the ratio of TIMP-1/MMP-9 expression (FIG. 7E-FIG. 7G).

### Example 13

### Discussion

Fruit consumption has been linked with improved lung function in asthma sufferers and the amelioration of acute airway inflammation in experimental models (16, 19, 40). However, no findings have been established in these studies in relation to Boysenberry compositions, airway fibrosis, or airway remodeling, and it is well established that other known asthma treatments have failed to address airway remodeling.

It is demonstrated herein that consumption of a Boysenberry composition moderates chronic lung remodeling and fibrosis in both a therapeutic and a prophylactic setting. Furthermore, the data indicate that macrophages play an important role in Boysenberry-mediated protection and that this protection may result from modulation of AAMs and increased MMP-9 activity.

An increase in both arginase activity (26, 27, 41) and AAMs (4, 9) is often linked with asthma pathogenesis. However, there is evidence that the presence of AAMs does not specifically underpin the development of allergic asthma (37), which indicates that AAMs may play an alternative role.

As shown herein, the Boysenberry treatment increased the population of arginase-positive AAMs alongside a drop in iNOS expression in the lung tissue of chronic OVA-challenged mice. Arginase and iNOS play an interactive role in regulating lung inflammation and repair (34, 35, 66). Where iNOS activity is associated with active inflammation, arginase expression is indicative of a switch toward inflammatory resolution (35, 63). Boysenberry consumption therefore appears to rebalance the lung environment, supporting inflammation resolution by modulating the functional phenotype of AAMs in the lung.

The presence of AAMs has been associated with decreased Th-2 cytokine production in lung inflammation (36, 42). However, it was determined that no changes in the levels of Th-2 cytokines IL-4, IL-5, and IL-13 with Boysenberry consumption following OVA challenge. This indicated that inhibition of proinflammatory Th-2 cytokine production by AAMs was not contributing to the protective effect of Boysenberry treatment.

Clinical and animal data indicate that the role of MMP-9 in asthma is multifaceted. Lung macrophages producing MMP-9 have been identified in both experimental and clinical settings (1, 5, 49). Elevated levels of active MMP-9 have been found in plasma and sputum samples from patients with asthma, compared with healthy controls (3, 23). Increased MMP-9 expression has been correlated with acute asthma exacerbation, including increased lung eosinophilia (6, 23). Conversely, an increase in MMP-9 levels has been associated with improved lung function in airway disease (25, 65). MMP-9 overexpression has also been shown to have beneficial effects in a model of pulmonary fibrosis (5). In contrast, data from MMP-9 knockout mice show a partial reduction in the development of asthma symptoms and reduced remodeling but, in some cases, a lack of MMP-9 has been shown to exacerbate disease (15, 24, 32).

MMP-9 exerts many downstream effects on different immune parameters, including the activation of both pro- and anti-inflammatory cytokines (15). Nevertheless, the data shown herein indicate that Boysenberry-induced protection of lung tissue from chronic collagen deposition and fibrosis is orchestrated, in part, through the generation of fibrolytic AAM producing MMP-9. Consistent with this, the data show that depletion of macrophages during the resolution phase of inflammation leads to increased collagen deposition with Boysenberry consumption. A similar resolution-promoting role for macrophages has been illustrated in bleomycin-induced pulmonary fibrosis (14).

Matrix metalloproteinases are regulated by their natural inhibitors TIMPs, and high TIMP-1/MMP-9 ratios are proposed to favour collagen deposition and lung remodeling (21, 28, 38). Here a significant increase was observed in the ratio of expression of TIMP-1/MMP-9 in the lung tissue of chronic OVA-challenged mice and this was reversed by Boysenberry treatment. The drop in the ratio of TIMP-1/MMP-9 in Boysenberry-treated mice therefore represents a potentially beneficial re-adjustment in the regulation of collagen deposition and breakdown.

TGFβ is associated with both normal (20) and pathological (17, 22, 56) tissue repair processes through its role in extracellular matrix production. In this study, it was observed that chronic OVA challenge led to a decrease in TGFβ levels that was reversed by Boysenberry consumption. There is evidence that TGFβ lowers the TIMP-1/MMP-9 ratio, thus favouring a more fibrolytic environment (18, 54, 56). As such the increase in TGFβ levels observed in the lungs of OVA-challenged mice following Boysenberry treatment could serve to limit excessive tissue fibrosis and inappropriate remodeling during lung repair by lowering the TIMP-1/MMP-9 ratio. TGFβ is also known to stimulate fibroblast contraction for normal tissue repair (20), which could likewise contribute toward the beneficial effects of Boysenberry treatment. As such the elevation of TGFβ has the potential to promote an anti-inflammatory, pro-resolution environment within the lung via multiple mechanisms.

The results from these studies show that Boysenberry administration exhibits a beneficial effect on chronic lung fibrosis in both a therapeutic and a prophylactic setting. This indicates that Boysenberry consumption may help avoid inappropriate fibrotic remodeling in cases of both poorly controlled and well-controlled asthma. Finally, these findings provide the first evidence that Boysenberry consumption could be used to support the development of fibrolytic AAMs with the potential to regulate appropriate lung remodeling in asthma and other lung conditions exhibiting fibrotic pathologies.

In summary, these findings have showed that Boysenberry compositions may be used to decrease inflammation and aberrant collagen deposition in the respiratory tract, and thereby find use in the treatment and prevention of various disorders of the airways, including asthma, chronic obstructive pulmonary disease, reactive airway disease, airway fibrosis, and airway remodeling.

Persons of ordinary skill can utilise the disclosures and teachings herein to produce other embodiments and variations without undue experimentation. All such embodiments and variations are considered to be part of this invention.

Accordingly, one of ordinary skill in the art will readily appreciate from the disclosure that later modifications, substitutions, and/or variations performing substantially the same function or achieving substantially the same result as embodiments described herein may be utilised according to such related embodiments of the present invention. Thus, the invention is intended to encompass, within its scope, the modifications, substitutions, and variations to processes, manufactures, compositions of matter, compounds, means, methods, and/or steps disclosed herein.

The description herein may contain subject matter that falls outside of the scope of the claimed invention. This subject matter is included to aid understanding of the invention.

In this specification, where reference has been made to external sources of information, including patent specifications and other documents, this is generally for the purpose of providing a context for discussing the features of the present invention. Unless stated otherwise, reference to such sources of information is not to be construed, in any jurisdiction, as an admission that such sources of information are prior art or form part of the common general knowledge in the art.

### REFERENCES

1. Atkinson J J, Lutey B A, Suzuki Y, Toennies H M, Kelley D G, Kobayashi D K, Ijem W G, Deslee G, Moore C H, Jacobs M E, Conradi S H, Gierada D S, Pierce R A, Betsuyaku T, Senior R M. The role of matrix metalloproteinase-9 in cigarette smoke-induced emphysema. Am J Respir Crit Care Med 183: 876-884, 2011.
2. Beamer C A, Migliaccio C T, Jessop F, Trapkus M, Yuan D, Holian A. Innate immune processes are sufficient for driving silicosis in mice. J Leukoc Biol 88: 547-557, 2010.
3. Belleguic C, Corbel M, Germain N, Lena H, Boichot E, Delaval P H, Lagente V. Increased release of matrix metalloproteinase-9 in the plasma of acute severe asthmatic patients. Clin Exp Allergy 32: 217-223, 2002.
4. Byers D E, Holtzman M J. Alternatively activated macrophages and airway disease. Chest 140: 768-774, 2011.
5. Cabrera S, Gaxiola M, Arreola J L, Ramirez R, Jara P, D'Armiento J, Richards T, Selman M, Pardo A. Overexpression of MMP9 in macrophages attenuates pulmonary fibrosis induced by bleomycin. Int J Biochem Cell Biol 39: 2324-2338, 2007.
6. Cataldo D D, Bettiol J, Noel A, Bartsch P, Foidart J M, Louis R. Matrix metalloproteinase-9, but not tissue inhibitor of matrix metalloproteinase-1, increases in the sputum from allergic asthmatic patients after allergen challenge. Chest 122: 1553-1559, 2002.
7. Cho J Y, Miller M, McElwain K, McElwain S, Shim J Y, Raz E, Broide D H. Remodeling associated expression of matrix metalloproteinase 9 but not tissue inhibitor of metalloproteinase 1 in airway epithelium: modulation by immunostimulatory DNA. J Allergy Clin Immunol 117: 618-625, 2006.
8. Corbel M, Belleguic C, Boichot E, Lagente V. Involvement of gelatinases (MMP-2 and MMP-9) in the development of airway inflammation and pulmonary fibrosis. Cell Biol Toxicol 18: 51-61, 2002.
9. Dasgupta P, Keegan A D. Contribution of alternatively activated macrophages to allergic lung inflammation: a tale of mice and men. J Innate Immun 4: 478-488, 2012.
10. Fireman E, Kraiem Z, Sade O, Greif J, Fireman Z. Induced sputum-retrieved matrix metalloproteinase 9 and tissue metalloproteinase inhibitor 1 in granulomatous diseases. Clin Exp Immunol 130: 331-337, 2002.
11. Forastiere F, Pistelli R, Sestini P, Fortes C, Renzoni E, Rusconi F, Dell'Orco V, Ciccone G, Bisanti L. Consumption of fresh fruit rich in vitamin C and wheezing symptoms in children. SIDRIA Collaborative Group, Italy (Italian Studies on Respiratory Disorders in Children and the Environment). Thorax 55: 283-288, 2000.
12. Fujita H, Aoki H, Ajioka I, Yamazaki M, Abe M, Oh-Nishi A, Sakimura K, Sugihara I. Detailed expression pattern of aldolase C (Aldoc) in the cerebellum, retina and other areas of the CNS studied in Aldoc-Venus knock-in mice. PLoS One 9: e86679, 2014.
13. Garcia V, Arts I C, Sterne J A, Thompson R L, Shaheen S O. Dietary intake of flavonoids and asthma in adults. Eur Respir J 26: 449-452, 2005.
14. Gibbons M A, MacKinnon A C, Ramachandran P, Dhaliwal K, Duffin R, Phythian-Adams A T, van Rooijen N, Haslett C, Howie S E, Simpson A J, Hirani N, Gauldie J, Iredale J P, Sethi T, Forbes S J. Ly6Chi monocytes direct alternatively activated profibrotic macrophage regulation of lung fibrosis. Am J Respir Crit Care Med 184: 569-581, 2011.
15. Greenlee K J, Corry D B, Engler D A, Matsunami R K, Tessier P, Cook R G, Werb Z, Kheradmand F. Proteomic identification of in vivo substrates for matrix metalloproteinases 2 and 9 reveals a mechanism for resolution of inflammation. J Immunol 177: 7312-7321, 2006.
16. Jang H Y, Kim S M, Yuk J E, Kwon O K, Oh S R, Lee H K, Jeong H, Ahn K S. Capsicum annuum L. methanolic extract inhibits ovalbumin-induced airway inflammation and oxidative stress in a mouse model of asthma. J Med Food 14: 1144-1151, 2011.
17. Kang H R, Cho S J, Lee C G, Homer R J, Elias J A. Transforming growth factor (TGF)-beta1 stimulates pulmonary fibrosis and inflammation via a Bax-dependent, bid-activated pathway that involves matrix metalloproteinase-12. J Biol Chem 282: 7723-7732, 2007.
18. Kaviratne M, Hesse M, Leusink M, Cheever A W, Davies S J, McKerrow J H Wakefield L M, Letterio J J, Wynn T A. IL-13 activates a mechanism of tissue fibrosis that is completely TGF-beta independent. J Immunol 173:4020-4029, 2004.
19. Kim S H, Kim B K, Lee Y C. Effects of Corni fructus on ovalbum-induced airway inflammation and airway hyper-responsiveness in a mouse model of allergic asthma. J Inflamm (Lond) 9: 9, 2012.
20. Kobayashi T, Kim H, Liu X, Sugiura H, Kohyama T, Fang Q, Wen F Q, Abe S, Wang X, Atkinson J J, Shipley J M, Senior R M, Rennard S I. Matrix metalloproteinase-9 activates TGF-beta and stimulates fibroblast contraction of collagen gels. Am J Physiol Lung Cell Mol Physiol 306: L1006-L1015, 2014.
21. Lagente V, Manoury B, Nenan S, Le Quement C, Martin-Chouly C, Boichot E. Role of matrix metalloproteinases in the development of airway inflammation and remodeling. Braz J Med Biol Res 38: 1521-1530, 2005.
22. Lee C G, Homer R J, Zhu Z, Lanone S, Wang X, Koteliansky V, Shipley J M, Gotwals P, Noble P, Chen Q, Senior R M, Elias J A. Interleukin-13 induces tissue fibrosis by selectively stimulating and activating transforming growth factor beta(1). J Exp Med 194: 809-821, 2001.
23. Lee Y C, Lee H B, Rhee Y K, Song C H. The involvement of matrix metalloproteinase-9 in airway inflammation of patients with acute asthma. Clin Exp Allergy 31: 1623-1630, 2001.
24. Lim D H, Cho J Y, Miller M, McElwain K, McElwain S, Broide D H. Reduced peribronchial fibrosis in allergen-challenged MMP-9-deficient mice. Am J Physiol Lung Cell Mol Physiol 291: L265-L271, 2006.
25. Lukkarinen H, Hogmalm A, Lappalainen U, Bry K. Matrix metalloproteinase-9 deficiency worsens lung injury in a model of bronchopulmonary dysplasia. Am J Respir Cell Mol Biol 41: 59-68, 2009.
26. Maarsingh H, Dekkers B G, Zuidhof A B, Bos I S, Menzen M H, Klein T, Flik G, Zaagsma J, Meurs H. Increased arginase activity contributes to airway remodeling in chronic allergic asthma. Eur Respir J 38:318-328, 2011.
27. Maarsingh H, Zaagsma J, Meurs H. Arginase: a key enzyme in the pathophysiology of allergic asthma opening novel therapeutic perspectives. Br J Pharmacol 158: 652-664, 2009.
28. Manoury B, Caulet-Maugendre S, Guenon I, Lagente V, Boichot E. TIMP-1 is a key factor of fibrogenic response to bleomycin in mouse lung. Int J Immunopathol Pharmacol 19: 471-487, 2006.
29. Martinez F O, Helming L, Gordon S. Alternative activation of macrophages: an immunologic functional perspective. Annu Rev Immunol 27:451-483, 2009.
30. Mauad T, Bel E H, Sterk P J. Asthma therapy and airway remodeling. J Allergy Clin Immunol 120: 997-1009; quiz 1010-1001, 2007.
31. McKinstry S U, Karadeniz Y B, Worthington A K, Hayrapetyan V Y, Ozlu M I, Serafin-Molina K, Risher W C, Ustunkaya T, Dragatsis I, Zeitlin S, Yin H H, Eroglu C. Huntingtin is required for normal excitatory synapse development in cortical and striatal circuits. J Neurosci 34:9455-9472, 2014.
32. McMillan S J, Kearley J, Campbell J D, Zhu X W, Larbi K Y, Shipley J M, Senior R M, Nourshargh S, Lloyd C M. Matrix metalloproteinase-9 deficiency results in enhanced allergen-induced airway inflammation. J Immunol 172: 2586-2594, 2004.
33. Mehra D, Sternberg D I, Jia Y, Canfield S, Lemaitre V, Nkyimbeng T, Wilder J, Sonett J, D'Armiento J. Altered lymphocyte trafficking and diminished airway reactivity in transgenic mice expressing human MMP-9 in a mouse model of asthma. Am J Physiol Lung Cell Mol Physiol 298:L189-L196, 2010.
34. Meurs H, Maarsingh H, Zaagsma J. Arginase and asthma: novel insights into nitric oxide homeostasis and airway hyperresponsiveness. Trends Pharmacol Sci 24: 450-455, 2003.
35. Mori M, Gotoh T. Regulation of nitric oxide production by arginine metabolic enzymes. Biochem Biophys Res Commun 275: 715-719, 2000.
36. Nair M G, Du Y, Perrigoue J G, Zaph C, Taylor J J, Goldschmidt M, Swain G P, Yancopoulos G D, Valenzuela D M, Murphy A, Karow M, Stevens S, Pearce E J, Artis D. Alternatively activated macrophage-derived RELM-α is a negative regulator of type 2 inflammation in the lung. J Exp Med 206: 937-952, 2009.
37. Nieuwenhuizen N E, Kirstein F, Jayakumar J, Emedi B, Hurdayal R, Horsnell W G, Lopata A L, Brombacher F. Allergic airway disease is unaffected by the absence of IL-4R alpha-dependent alternatively activated macrophages. J Allergy Clin Immunol 130: 743-750.e8, 2012.
38. Ohbayashi H, Shimokata K. Matrix metalloproteinase-9 and airway remodeling in asthma. Curr Drug Targets Inflamm Allergy 4: 177-181, 2005.
39. Okoko B J, Burney P G, Newson R B, Potts J F, Shaheen S O. Childhood asthma and fruit consumption. Eur Respir J 29: 1161-1168, 2007.
40. Park S J, Shin W H, Seo J W, Kim E J. Anthocyanins inhibit airway inflammation and hyperresponsiveness in a murine asthma model. Food Chem Toxicol 45: 1459-1467, 2007.
41. Pera T, Zuidhof A B, Smit M, Menzen M H, Klein T, Flik G, Zaagsma J, Meurs H, Maarsingh H. Arginase inhibition prevents inflammation and remodeling in a guinea pig model of chronic obstructive pulmonary disease. J Pharmacol Exp Ther 349: 229-238, 2014.
42. Pesce J T, Ramalingam T R, Mentink-Kane M M, Wilson M S, El Kasmi K C, Smith A M, Thompson R W, Cheever A W, Murray P J, Wynn T A. Arginase-1-expressing macrophages suppress Th2 cytokine-driven inflammation and fibrosis. PLoS Pathog 5: e1000371, 2009.
43. Peters S P. Asthma treatment in the 21st century: what's next? Clin Rev Allergy Immunol 27: 197-205, 2004.
44. Priceman S J, Sung J L, Shaposhnik Z, Burton J B, Torres-Collado A X, Moughon D L, Johnson M, Lusis A J, Cohen D A, Iruela-Arispe M L, Wu L. Targeting distinct tumor-infiltrating myeloid cells by inhibiting CSF-1 receptor: combating tumor evasion of antiangiogenic therapy. Blood 115: 1461-1471, 2010.
45. Roche W R, Beasley R, Williams J H, Holgate S T. Subepithelial fibrosis in the bronchi of asthmatics. Lancet 1: 520-524, 1989.
46. Romieu I, Varraso R, Avenel V, Leynaert B, Kauffmann F, Clavel-Chapelon F. Fruit and vegetable intakes and asthma in the E3N study. Thorax 61: 209-215, 2006.
47. Rosenlund H, Kull I, Pershagen G, Wolk A, Wickman M, Bergstrom A. Fruit and vegetable consumption in relation to allergy: disease-related modification of consumption? J Allergy Clin Immunol 127: 1219-1225, 2011.
48. Rosenlund H, Magnusson J, Kull I, Hakansson N, Wolk A, Pershagen G, Wickman M, Bergstrom A. Antioxidant intake and allergic disease in children. Clin Exp Allergy 42: 1491-1500, 2012.
49. Russell R E, Culpitt S V, DeMatos C, Donnelly L, Smith M, Wiggins J, Barnes P J. Release and activity of matrix metalloproteinase-9 and tissue inhibitor of metalloproteinase-1 by alveolar macrophages from patients with chronic obstructive pulmonary disease. Am J Respir Cell Mol Biol 26: 602-609, 2002.
50. Schneider C A, Rasband W S, Eliceiri K W. NIH Image to ImageJ: 25 years of image analysis. Nat Methods 9: 671-675, 2012.
51. Shaheen S O, Sterne J A, Thompson R L, Songhurst C E, Margetts B M, Burney P G. Dietary antioxidants and asthma in adults: population-based case-control study. Am J Respir Crit Care Med 164: 1823-1828, 2001.
52. Shaw O M, Harper J L. An efficient single prime protocol for the induction of antigen-induced airways inflammation. J Immunol Methods 395: 79-82, 2013.
53. Sin Y Y, Ballantyne L L, Mukherjee K, St Amand T, Kyriakopoulou L, Schulze A, Funk C D. Inducible arginase 1 deficiency in mice leads to hyperargininemia and altered amino acid metabolism. PLoS One 8:e80001, 2013.
54. Todorova L, Gurcan E, Westergren-Thorsson G, Miller-Larsson A. Budesonide/formoterol effects on metalloproteolytic balance in TGFbeta-activated human lung fibroblasts. Respir Med 103: 1755-1763, 2009.
55. Urso M L, Wang R, Zambraski E J, Liang B T. Adenosine A3 receptor stimulation reduces muscle injury following physical trauma and is associated with alterations in the MMP/TIMP response. J Appl Physiol 112:658-670, 2012.
56. Van Bruaene N, Derycke L, Perez-Novo C A, Gevaert P, Holtappels G, De Ruyck N, Cuvelier C, Van Cauwenberge P, Bachert C. TGF-beta signaling and collagen deposition in chronic rhinosinusitis. J Allergy Clin Immunol 124: 253-259, 259.e1-e2, 2009.
57. van den Hengel L G, Hellingman A A, Nossent A Y, van Oeveren-Rietdijk A M, de Vries M R, Spek C A, van Zonneveld A J, Reitsma P H, Hamming J F, de Boer H C, Versteeg H H, Quax P H. Protease-activated receptor (PAR)2, but not PAR1, is involved in collateral formation and anti-inflammatory monocyte polarization in a mouse hind limb ischemia model. PLoS One 8: e61923, 2013.
58. Van Rooijen N, Sanders A. Liposome mediated depletion of macrophages: mechanism of action, preparation of liposomes and applications. J Immunol Methods 174: 83-93, 1994.
59. Vignola A M, Kips J, Bousquet J. Tissue remodeling as a feature of persistent asthma. J Allergy Clin Immunol 105: 1041-1053, 2000.
60. Weidenbusch M, Anders H J. Tissue microenvironments define and get reinforced by macrophage phenotypes in homeostasis or during inflammation, repair and fibrosis. J Innate Immun 4: 463-477, 2012.
61. WHO. Prevention of Allergy and Allergic Asthma: Based on the WHO/WAO Meeting on the Prevention of Allergy and Allergic Asthma, Geneva, 8-9 Jan. 2002. Geneva: World Health Organization, 2003.
62. Woods R K, Walters E H, Raven J M, Wolfe R, Ireland P D, Thien F C K, Abramson M J. Food and nutrient intakes and asthma risk in young adults. Am J Clin Nutr 78: 414-421, 2003.
63. Wu G, Morris S M Jr. Arginine metabolism: nitric oxide and beyond. Biochem J 336: 1-17, 1998.
64. Wu K, Koo J, Jiang X, Chen R, Cohen S N, Nathan C. Improved control of tuberculosis and activation of macrophages in mice lacking protein kinase R. PLoS One 7: e30512, 2012.
65. Yoon H K, Cho H Y, Kleeberger S R. Protective role of matrix metalloproteinase-9 in ozone-induced airway inflammation. Environ Health Perspect 115: 1557-1563, 2007.
66. Zimmermann N, Rothenberg M E. The arginine-arginase balance in asthma and lung inflammation. Eur J Pharmacol 533: 253-262, 2006.
67. Lieberman P L, Oppenheimer J, Desai M, 2015, Allergic Remodeling, World Allergy Organisation, article published online at: http://www.worldallergy.org/professional/allergic_diseases center/allergic_remodeling/
68. Holgate S T, Holloway J, Wilson S, Bucchieri F, Puddicombe S, Davies D E. Epithelial-mesenchymal communication in the pathogenesis of chronic asthma. Proc Am Thorac Soc. 1(2):93-98, 2004.
69. Chakir J, Shannon J, Molet S, et al. Airway remodeling-associated mediators in moderate to severe asthma: effect of steroids on TGF-beta, IL-11, IL-17, and type I and type III collagen expression. J Allergy Clin Immunol. 111(6):1293-1298, 2003.
70. The Childhood Asthma Management Program Research Group. Long-term effects of budesonide or nedocromil in children with asthma. N Engl J Med. 343(15):1054-1063, 2000.
71. Busse W W, Pedersen S, Pauwels R A, et al. The Inhaled Steroid Treatment As Regular Therapy in Early Asthma (START) study 5-year follow-up: effectiveness of early intervention with budesonide in mild persistent asthma. J Allergy Clin Immunol. 121(5):1167-1174, 2008.
72. Covar R A, Spahn J D, Murphy J R, Szefler S J, Group CAMPR. Progression of asthma measured by lung function in the childhood asthma management program. Am J Respir Crit Care Med. 170(3):234-241, 2004.
73. Guilbert T W, Morgan W J, Zeiger R S, et al. Long-term inhaled corticosteroids in preschool children at high risk for asthma. N Engl J Med. 354(19):1985-1997, 2006.
74. Shaw O M, Hurst R D, Harper J L, Boysenberry ingestion supports fibrolytic macrophages with the capacity to ameliorate chronic lung remodeling, Am J Physiol Lung Cell Mol Physiol 311: L628-L638, 2016.
75. Singleton, Vernon L, Orthofer, Rudolf, Lamuela-Raventós, Rosa M. Analysis of total phenols and other oxidation substrates and antioxidants by means of Folin-Ciocalteu reagent 299: 152, 1999.
76. Lister C E, Lancaster J E, Sutton K H, Walker J R L. Development changes in the concentration and composition of flavonoids in skin of a red and a green apple cultivar. Journal of the Science of Food and Agriculture 64: 155-161, 1994.
77. Cao G, Alessio H, Cutler R. Oxygen-radical absorbance capacity assay for antioxidants. Free Radical Biology and Medicine 14 (3): 303-311, 1993.
78. Ou B, Hampsch-Woodill M, Prior R. Development and validation of an improved oxygen radical absorbance capacity assay using fluorescein as the fluorescent probe. Journal of Agricultural and Food Chemistry 49 (10): 4619-4626, 2001.
79. Brand-Williams W, Cuvelier M E, Berset C. Use of a free radical method to evaluate antioxidant activity. Lebensm. Wiss. Tecnol. 28: 25-30, 1995.
80. Sanchez-Moreno C, Larraui J A, Saura-Calixto F. A procedure to measure the antiradical efficiency of polyphenols. Journal of Science and the Food of Agriculture 76: 270-276, 1998.
81. Sun-Waterhouse D, Wen I, Wibisono R, Melton L D, Wadhwa S. Evaluation of the extraction efficiency for polyphenol extracts from by-products of green kiwifruit juicing. International Journal of Food Science & Technology. 44(12): 2644-2652, 2009.
82. Eidenberger T, Selg M, Fuerst S, Krennhuber K. In-vitro inhibition of human lipase PS by polyphenols from kiwi fruit. Journal of Food Research. 3(4): 71-77, 2014.
83. Athar N, McLaughlin J, Taylor G. The concise New Zealand food composition tables. 6th edition. New Zealand Institute for Crop & Food Research/Ministry of Health: Palmerston North, New Zealand. 177 p, 2003.
84. Wada L, Ou B. Antioxidant activity and phenolic content of Oregon caneberries. Journal of Agricultural and Food Chemistry 50: 3495-3500, 2002.
85. Lister C E, Andrews F M, Ganeshan D. Comparison of Chilean and New Zealand boysenberry fruit and concentrates, Crop & Food Research Report No. 2059, 2008.
86. Terzikhan N, et al. Prevalence and incidence of COPD in smokers and non-smokers: the Rotterdam Study Eur J Epidemiol 31:785-792, 2016.

What is claimed is:

1. A method for:
(i) treating or preventing inflammation in the respiratory tract of a subject;
(ii) treating or preventing asthma in a subject; or
(iii) treating or preventing chronic obstructive pulmonary disease in a subject;
comprising administering to the subject a composition comprising a Boysenberry concentrate, thereby treating or preventing the inflammation in the respiratory tract, asthma, or chronic obstructive pulmonary disease in the subject.

2. The method of claim 1, wherein the composition comprises Boysenberry juice concentrate or a Boysenberry powder.

3. The method of claim 1, wherein the composition comprises a dosage unit comprising about 5 to about 500 mg total anthocyanins.

4. The method of claim 1, wherein the composition is administered by enteral or oral administration.

5. The method of claim 1, wherein the composition is administered as formulation selected from the group consisting of a syrup, drops, gel, jelly, tablet, and capsule.

6. The method of claim 1, wherein the composition is administered to obtain
(i) a dosage of about 0.1 mg/kg to about 10 mg/kg total anthocyanins/subject's body weight; or
(ii) a dosage of about 10 mg to about 1000 mg total anthocyanins per day.

7. The method of claim 1, wherein the composition comprises added polyphenols.

8. The method of claim 1, wherein the composition is co-administered with a further respiratory aid.

9. The method of claim 1, wherein the inflammation is associated with one or more of:
(i) a chronic respiratory disorder;
(ii) reactive airway disease;
(iii) airway fibrosis; and/or
(iiv) airway remodeling.

10. A method for:
(i) treating or preventing aberrant collagen deposition in the respiratory tract of a subject;
(ii) treating or preventing fibrosis in the respiratory tract of a subject; or
(iii) treating or preventing airway remodeling in the respiratory tract of a subject;
comprising administering to the subject a composition comprising a Boysenberry concentrate, thereby treating or preventing the aberrant collagen deposition in the respiratory tract, fibrosis in the respiratory tract, or airway remodeling in the subject.

11. The method of claim 10, wherein the composition comprises Boysenberry juice concentrate or a Boysenberry powder.

12. The method of claim 10, wherein the composition comprises a dosage unit comprising about 5 to about 500 mg total anthocyanins.

13. The method of claim 10, wherein the composition is administered by enteral or oral administration.

14. The method of claim 10, wherein the composition is administered as a formulation selected from the group consisting of a syrup, drops, gel, jelly, tablet, and capsule.

15. The method of claim 10, wherein the composition administered to obtain:
(i) a dosage of about 0.1 mg/kg to about 10 mg/kg total anthocyanins/subject's body weight; or
(ii) a dosage of about 10 mg to about 1000 mg total anthocyanins per day.

16. The method of claim 10, wherein the composition comprises added polyphenols.

17. The method of claim 10, wherein the composition is co-administered with a further respiratory aid.

18. The method of claim 17, wherein the further respiratory aid is selected from the group consisting of a bronchodilator, corticosteroid, and anti-inflammatory agent.

19. The method of claim 10, wherein the fibrosis or the airway remodeling is associated with a chronic respiratory disorder.

20. The method of claim 19, wherein the further respiratory disorder is selected from the group consisting of asthma, chronic obstructive pulmonary disease, and reactive airway disease.
